(19) United States
(12) Patent Application Publication
(10) Pub. No.: US 2012/0121610 A1
(43) Pub. Date: May 17, 2012

(54) THERAPEUTIC AGENT AND ASSAY

(76) Inventors: Carl Smythe, Sheffield (GB); Richard Beniston, Sheffield (GB)

(21) Appl. No.: 13/376,954

(22) PCT Filed: Jun. 10, 2010
(86) PCT No.: PCT/GB10/50976

§ 371 Date: Jan. 31, 2012

(30) Foreign Application Priority Data
Jun. 10, 2009 (GB) 0909944.1

(51) Int. Cl. A61K 39/395; A61P 35/00; A61K 31/7105; C12Q 1/68; C07K 16/18; C07H 21/02

(52) U.S. Cl. 424/172.1; 530/389.8; 536/24.5; 514/44 A; 514/34; 435/6.1

(57) ABSTRACT

The present invention relates to an agent that is useful in the treatment of a cell proliferative disease or disorder, and an assay for identifying such an agent.

The present invention relates to an agent that is useful in the treatment of a cell proliferative disease or disorder, and an assay for identifying such an agent.

Cell cycle checkpoints are believed to comprise surveillance mechanisms which act to prevent the replication of damaged DNA or segregation of damaged chromosomes. Their importance in maintaining genomic stability is underscored by genetic disorders such as ataxia telangiectasia and Nijmegen breakage syndrome which are associated with checkpoint deficiencies and characterised by genetic instability and a high incidence of tumours. Most recently it has become clear that checkpoint genes originally identified by virtue of a defective cell cycle phenotype, participate more widely to affect a co-ordinated cellular response to the presence of chromosomal lesions, although the relationship between cell cycle arrest and progression of DNA repair is not well established.

The mammalian serine/threonine protein kinase Chk1 has previously been implicated in controlling the G2/M checkpoint in mammalian cells. In recent studies, the present inventors have shown that Chk1 is a signalling component of pathways which sense arrested replication forks and ionising radiation-induced double strand breaks and that one function of Chk1 is to ensure that activation of late-firing replication origins is blocked when synthesis from origins firing early in S phase is inhibited. Importantly, it has been found that Chk1 also acts to stabilise components of the replication machinery when replication fork progress is slowed or impeded. The absence of Chk1 function in cells with slow moving or arrested replication forks causes replication fork abandonment. Where arrested replication forks lose the capacity for further elongation, replication proteins dissociate from sites of synthesis.

Activation of Chk1 in response to DNA damage or replication arrest is known to involve its phosphorylation at multiple residues, by the DNA activated PIK kinase family members ATM (DNA damage) and ATR (replication arrest). ATM or ATR are necessary for Chk1 phosphorylation in vitro and in vivo, however they are not sufficient for efficient and timely Chk1 phosphorylation in vivo.

Claspin was originally identified as a protein in Xenopus egg extracts which bound tightly to Chk1, and which when depleted resulted in failure to bring about ATR-mediated phosphorylation and activation of Chk1 in the presence of the replication inhibitor aphidicolin. Claspin is loaded onto DNA replication origins during replication initiation, and is postulated to bind at the time of initial origin unwinding but prior to the initiation of DNA synthesis. Studies in Xenopus egg extracts indicate Claspin loading requires the presence of Cdc45, a factor that promotes the initial unwinding of the origin DNA in the presence of Cdk2. This step is followed by RPA binding which is a prerequisite for recruitment of PCNA and DNA polymerases alpha and delta. As RPA is not required for Claspin binding, it is postulated that Claspin binds at the time of initial origin unwinding but prior to the initiation of DNA synthesis. Claspin is believed to associate with replication fork machinery where it may serve as a checkpoint sensor protein. Even though associated with the replication fork, Claspin is not an essential DNA replication factor.

Germline mutations in BRCA1 are responsible for many cases of hereditary breast cancer. One defective germline copy of BRCA1 causes cancer predisposition, while the second allele is consistently lost in cells from tumours in predisposed individuals. Cells deficient in BRCA1 sustain spontaneous aberrations in chromosome structure. Such findings indicate that BRCA1 is essential for preserving chromosome structure and thus suppressing genome instability. However despite considerable work over many years, the mechanisms by which BRCA1 acts to maintain genomic stability remains unclear.

Recent evidence indicates that gross chromosomal arrangements which emerge in BRCA1-deficient cells may result not from a failure of DNA repair but rather from inappropriate repair of lesions that occur both during and as a consequence of aberrant events within S-phase. BRCA1-deficient rodent cells and human tumours are deficient in homologous recombination, a repair pathway which is potentially error-free during S-phase, while retaining the ability to carry out error-prone non-homologous end joining. Thus spontaneous or induced chromosomal lesions in BRCA1-deficient cells may be re-routed for repair by error-prone mechanisms because the preferred route of processing is unavailable.

Breast cancer is the leading cause of cancer-related mortality among women worldwide. The current market size of drugs used for the treatment of breast cancer is estimated at $ 3.3 billion. 5-10% of all breast cancers in the US are associated with an inherited genetic abnormality. The most common genetic abnormalities involve the genes BRCA1 and BRCA2.

Many breast tumours over-express hormone or growth receptors and substantial investment has been made into the development of appropriate receptor antagonists/inhibitors. Significant recent developments include the successful introduction to market of the receptor antagonist herceptin (e.g. Genentech's Herceptin and Genentech/Roche's Avastin (bevacizumab)), which has significant efficacy against sporadic tumours whose proliferation is dependent on the presence of HER2 proteins. GlaxoSmithKline's′ Tykerb (lapatinib, a dual tyrosine kinase inhibitor) will expand treatment options for HER-2 positive, Herceptin-refractory, locally advanced and metastatic patients and was FDA approved in March 2007. As an estimated 40 of patients are Herceptin-refractory, use of Tykerb will become significant. As of 2006, there are at least 144 candidates in the breast cancer pipeline of which, 26 are molecular-targeted therapeutics (MTTs) which are expected to inactivate hormone receptors or recently identified downstream components. Due to the cytostatic nature of several MTTs, significant tumour regressions will likely be achieved when combined with cytotoxics. Many cytotoxics in development are reformulations of those currently on the market, in an attempt to decrease toxicity levels and thus the harsh side effects often associated with their use, or to ease their administration. High unmet needs still persist for so-called “receptor-positive” tumours. Similar to numerous forms of cancer, breast tumour growth is a multifactor disease with no standardised medication available for patients. Survival rates are variable and in the metastatic setting, the overall survival rate remains below 5 years. Cytotoxics and antihormonals serve only to slow the progress of metastatic disease. Curative treatments with lower levels of toxicity for patients with metastatic disease are urgently needed and the advent of drugs with highly specific targets could potentially change this situation. Additionally, therapies tailored to the patient genotype would be advantageous.

Further, despite the identification over 12 years ago of BRCA1 and BRCA2 as genes which cause hereditary breast cancer, there has been little progress in the development of treatments for individuals with an inherited predisposition to this disease. Such individuals constitute 5-10 of all breast cancer cases. This lack of progress is largely due to: (i) a continued lack of understanding of how mutated forms of these genes cause a cell to become transformed; and (ii) the absence to date of a coherent strategy to exploit the inherent biological difference between normal and cancer cells in mammary tissue in order to kill the latter while sparing the former.

Breast cancers in women with BRCA1 abnormalities are predominantly receptor-negative and have high-grade cell growth indicating a very high unmet need. Both of these characteristics mean that preventative mastectomy, traditional chemotherapy, or combinations of chemotherapy and mastectomy remain the current principal approaches in treating these patients. Such approaches have significant limitations for patient survival, and have a substantial impact on patient quality of life, given the toxicity of current chemotherapy and the relatively obvious psycho-social impact of the surgical approach. Despite the prospect that identification of genes involved in hereditary breast cancer would lead to potential therapeutics, few, if any, obvious pipelines exist and thus there is a substantial potential market for new drugs which might be expected to exploit genetic status to achieve a therapeutic impact in a significant element of the overall market.

The present inventors have identified a new role for a gene product termed Claspin, whose function is to co-operate with BRCA1 in maintaining the viability of cells during the critical process of DNA replication. Individuals who are predisposed to the development of cancer (and in particular breast cancer) have inherited one functional and one non-functional form of either BRCA1 or BRCA2. Such individuals are said to be heterozygous carriers for the relevant BRCA gene. The current estimate of heterozygous carriers in the general Caucasian population is about one in 1000 for BRCA1 and recent analysis on 22 population-based & hospital-based studies showed the average cumulative risks in BRCA1-mutation carriers by the age of 70 was 65% for breast cancer and 39% for ovarian cancer, in addition to an increased risk of other cancers such as: colon, cervix, uterus, pancreas and prostate.

During the process of transformation, which will lead ultimately to the onset of the disease, cells emerge which have lost the second functional form of the relevant BRCA gene.

The present inventors have found that cancer cells lacking both copies of BRCA1 and lacking Claspin are profoundly sensitive to agents used in chemotherapeutic intervention (such as agents which temporarily interfere with DNA replication). The data indicate that in such circumstances, these cells are inviable, fail to undergo any further proliferation and die. In contrast, cells which retain either Claspin, or a normal copy of BRCA1, are relatively insensitive to such treatment.

It follows that, in cancer patients (e.g. hereditary breast cancer patients), a combination therapy comprising anti-tumour agents (such as inhibitors of DNA replication) in combination with a specific Claspin inhibitor would be expected to have a high therapeutic index (and certainly higher than existing therapies), by selectively blocking the proliferation of those cells that lack both copies of BRCA1 (i.e. in a tumour), while having minimal effects on healthy surrounding tissues, which retain one functional copy of the BRCA1 gene.

In the present invention, it should be understood that any reference to “BRCA” in the claims or description is reference to one or both forms of BRCA, namely BRCA1 and BRCA2. Preferably, BRCA1 is the form of BRCA of primary interest.

Thus, the present invention relates to the identification of a potential drug target, the identification of a lead molecule (e.g. an siRNA-based lead molecule) with efficacy in the ablation of the target, and an assay which is suitable for identifying additional conventional small molecule agents which may be useful in the treatment of cancer (e.g. breast cancer).

The present inventors have established a rapid and robust assay which enables evaluation of the ability of compounds to interfere with this role of Claspin and BRCA1. Together with the knowledge of known roles of these proteins, the present invention enables the unique position to be able to screen compound libraries, some compounds of which might be expected to interfere with relevant functions related to chromosomal integrity and as a consequence interfere with tumour specific growth.

Further, the present inventors have identified a small molecule which is capable of blocking Claspin function, thus acting as a lead compound which could be used in the development of a clinical lead.

Thus, in one embodiment of the present invention, there is provided a modulator of claspin for use in the treatment of a cell proliferative disorder.

In another aspect, there is provided a modulator of claspin in combination with at least one anti-tumour agent for use in the treatment of a cell proliferative disorder.

The combination of the modulator of claspin with the anti-tumour agent during said treatment can be combined, sequential or simultaneous.

Preferably, the cell proliferative disorder is associated with impaired BRCA function, such as a cancer, especially but not solely breast cancer. Thus, the modulator of the present invention can be used in the treatment of an animal predetermined to have cancer.

In some aspects of the present invention, the modulator of claspin is a claspin-specific antibody or a claspin-specific nucleic acid modulator. Preferably the modulator modulates the activity or expression of claspin. Preferably the modulator is an inhibitor, such as an RNA inhibitor or an antisense oligomer.

In another embodiment of the present invention, there is provided a use of a modulator of claspin together with an anti-tumour agent in the manufacture of a medicament containing the modulator and the anti-tumour agent for combined, sequential or simultaneous administration for the treatment of a cell proliferative disease.

Also provided is a pharmaceutical composition comprising a modulator of claspin. Optionally, the pharmaceutical composition may further contain an anti-tumour agent, and/or other physiologically acceptable excipients or adjuvants.

In embodiments of the present invention where an anti-tumour agent may be used, the anti-tumour agent can be an agent selected from the group consisting of: methotrexate, 5-fluorouracil, fluorodeoxyuridine, cytosine arabinoside, 6-mercaptopurine, 6-thioguanine, mechloroethamine, cyclophosphamide, ifosfamide, melphalan, chlorambucil, thiotepa, mitomycin C, aziridinylbenzoquinone (AZQ), busulfan, carmustine (BCNU), lomustine (CCNU), fotemustine, carboplatin, daunorubicin, doxorubicin or adriamycin, epirubicin, dactinomycin or actinomycin D, mitoxanthrone, amsacrine, tenoposide, etoposide, irinotecan, topotecan, vincristine, vinblastine, vindesine, vinorelbine, taxol, taxotere, and mixtures thereof.

In another embodiment of the present invention, there is provided a purified inhibitor of claspin. Preferably such an inhibitor is a claspin-specific antibody or a claspin-specific nucleic acid modulator.

This invention relates to compounds, compositions, and methods useful for modulating the expression and activity of e.g. claspin by RNA interference (RNAi) using small nucleic acid molecules. In particular, the instant invention features small nucleic acid molecules, such as short interfering nucleic acid (siNA), short interfering RNA (sRNA), double-stranded RNA (dsRNA), micro-RNA (miRNA), and short hairpin RNA (shRNA) molecules and methods used to modulate the expression of claspin genes.

A siNA of the invention can be unmodified or chemically-modified. A siNA of the instant invention can be chemically synthesized, expressed from a vector or enzymatically synthesized.

In one embodiment, the invention features a double-stranded short interfering nucleic acid (siNA) molecule that down-regulates expression of a claspin gene, wherein said siNA molecule comprises about 15 to about 28 base pairs.

In one embodiment, the invention features a double stranded short interfering nucleic acid (siNA) molecule that directs cleavage of a claspin RNA via RNA interference (RNAi), wherein the double stranded siNA molecule comprises a first and a second strand, each strand of the siNA molecule is about 18 to about 28 nucleotides in length, the first strand of the siNA molecule comprises nucleotide sequence having sufficient complementarity to the claspin RNA for the siNA molecule to direct cleavage of the claspin RNA via RNA interference, and the second strand of said siNA molecule comprises nucleotide sequence that is complementary to the first strand.

In one embodiment of the invention a siNA molecule comprises an antisense strand comprising a nucleotide sequence that is complementary to an mRNA sequence or a portion thereof encoding a claspin protein. Optionally, the siNA further comprises a sense strand, wherein said sense strand is complementary to said antisense strand.

In one embodiment, the antisense region of claspin siNA constructs comprises a sequence complementary to sequence having any of SEQ ID NOs. 1, 3, 5, and 7. In one embodiment, the antisense region of claspin constructs comprises sequence having any of SEQ ID NOs. 2, 4, 6, 8, 9, 10, 11, 12 and 13. In another embodiment, where siNA duplexes are provided, the sense region of claspin constructs comprises a sequence having any of SEQ ID NOs. 1, 3, 5, 7, and complementary sequences to SEQ ID NOs: 9-13. In one embodiment, a siNA molecule of the invention comprises any of SEQ ID NOs. 1-13.

In one embodiment of the invention, there is provided an RNA comprising, or consisting of, or consisting essentially of, a sequence of SEQ ID NO: 2, 4, 6, 8, 9, 10, 11, 12 or 13. Preferably, the sequence is one of SEQ ID NOs: 9, 10, 11, 12 or 13.

In some embodiments, there may be provided a sequence of SEQ ID NOs: 2, 4, 6, 8, 9, 10, 11, 12, or 13 with various base substitutions, additions or deletions to said sequence. Preferably any substitutions, additions or deletions will be such that the function of the RNA as an antisense molecule to the claspin mRNA will not be significantly compromised. Preferably, the number of bases to be added, deleted or substituted will be approximately only 1, 2 or 3 bases. The ability of an antisense molecule to remain a viable antisense molecule can be tested via e.g. hybridisation tests under various stringencies, as described in more detail below.

In a preferred aspect, any additional bases to the RNA antisense molecule will be complementary to the claspin mRNA.

In one embodiment of the invention a siNA molecule comprises an antisense strand having about 15 to about 30 (e.g. about 15, 16, 17, 18, 19, 20, 21, 22, 23, 24, 25, 26, 27, 28, 29 or 30) nucleotides, wherein the antisense strand is complementary to a RNA sequence or a portion thereof encoding a claspin protein, and wherein optionally_said siNA further comprises a sense strand having about 15 to about 30 (e.g. about 15, 16, 17, 18, 19, 20, 21, 22, 23, 24, 25, 26, 27, 28, 29 or 30) nucleotides, and wherein said sense strand and said antisense strand are distinct nucleotide sequences where at least about 15 nucleotides in each strand are complementary to the other strand.

In other embodiments of the invention, there is provided a siNA molecule having about 21 nucleotides in an antisense strand, optionally +/−1 or 2 nucleotides at the 5′ and/or 3′ end of the sequence.

Methods of manufacturing siNA duplexes are well known to those of skill in the art. For example, the nucleic acid molecules of the present invention can be synthesized separately and joined together post-synthetically, for example, by ligation (Moore et al., 1992, Science 256,9923; Draper et al., International PCT publication No. WO 93/23569; Shabarova et al., 1991, Nucleic Acids Research 19,4247; Bellon et al., 1997, Nucleosides & Nucleotides, 16,951; Bellon et al., 1997, Bioconjugate Clje7n. 8, 204), or by hybridization following synthesis and/or deprotection.

The siNA molecules of the invention can also be synthesized via a tandem synthesis methodology, wherein both siNA strands are synthesized as a single contiguous oligonucleotide fragment or strand separated by a cleavable linker which is subsequently cleaved to provide separate siNA fragments or strands that hybridize and permit purification of the siNA duplex. The linker can be a polynucleotide linker or a non-nucleotide linker. The tandem synthesis of siNA can be readily adapted to both multiwell/multiplate synthesis platforms such as 96 well or similarly larger multi-well platforms. The tandem synthesis of siNA can also be readily adapted to large scale synthesis platforms employing batch reactors, synthesis columns and the like.

A siNA molecule can also be assembled from two distinct nucleic acid strands or fragments wherein one fragment includes the sense region and the second fragment includes the antisense region of the RNA molecule.

In the present invention, the inhibitor may be a claspin-specific nucleic acid modulator with any of the following sequences:

```
              (SEQ ID NO: 1)
    5′-GCAGAUGGGUUCUUAAAUGUU-3′
          +++++++++++++++++++
3′-UUCGUCUACCCAAGAAUUUAC-5′
              (SEQ ID NO: 2)
              (SEQ ID NO: 3)
    5′-GAGUAGAUGUUUCCAUUAAUU-3′
          +++++++++++++++++++
3′-UUCUCAUCUACAAAGGUAAUU-5′
              (SEQ ID NO: 4)
              (SEQ ID NO: 5)
    5′-GCAGAUAGUCCUUCAGAUAUU-3′
          +++++++++++++++++++
3′-UUCGUCUAUCAGGAAGUCUAU-5′
              (SEQ ID NO: 6)
              (SEQ ID NO: 7)
    5′-GAAGACAGGCUCACUGCUAUU-3′
          +++++++++++++++++++
3′-UUCUUCUGUCCGAGUGACGAU-5′
              (SEQ ID NO: 8)
```

In one embodiment of the present invention, one or more of the siRNA duplexes are used to inhibit claspin.

In another embodiment, one or more of the siRNA antisense strands (SEQ ID NOs: 2, 4, 6, 8) are used to inhibit claspin.

In a further embodiment of the present invention, a mixture of each of the 4 different siRNA duplex molecules are used to ablate claspin.

In a preferred embodiment of the present invention, there is provided a sequence comprising SEQ ID NOs: 9, 10, 11, 12 or 13. One or more of those sequences, alone or in combination, can be used to inhibit claspin function.

(SEQ ID NO: 9)
5′-GACAGUGAUUCCGAAACAGUU-3′
(SEQ ID NO: 10)
5′-GCAACUGGGAGUAGAUGUUUU-3′
(SEQ ID NO: 11)
5′-CAGAUGAAAACUCAGGCAAUU-3′
(SEQ ID NO: 12)
5′-GUUGAAAAGGCAAAUGAGGUU-3′
(SEQ ID NO: 13)
5′-UCGUCUAAGUCUCAGGUAAUU-3′

In a preferred embodiment, there are also provided duplexes comprising a sequence of SEQ ID NO: 9 to 13 and their respective complementary sequences.

In a preferred embodiment of the present invention, there is provided an in vitro method of identifying a claspin modulating agent, or an agent that modulates a downstream component involved in the Claspin-dependent control of the DNA replication process, said method comprising the steps of:
(a) providing an assay system comprising a claspin polypeptide or nucleic acid or a functionally active fragment or derivative thereof;
(b) contacting the assay system with a test agent; and
(c) detecting the expression or activity of claspin in the assay system, wherein a difference between the expression or activity of claspin in the presence of the test agent compared to in its absence identifies the test agent as a claspin modulating agent.

In this regard, the assay is preferably used to determine whether a test agent is an inhibitor of claspin. In order to determine whether an agent is a modulator of claspin, preferably the assay system comprises cultured cells that express the claspin polypeptide. Non-exhaustive examples of such cells are HeLa, U2OS cells or MCF-7 cells. Optionally, the cells may be heterozygous for BRCA1 (e.g. MDA-MB-231 cells) or BRCA-null cells (e.g. HCC1937, SUM1315MO2). In some embodiments, the cells may be BRCA-heterozygous or null cells which have additionally been subjected to ablation of claspin.

In one embodiment, the function of the claspin (e.g. the expression and/or activity of the claspin) in the assay system before the addition of a test agent is measured by various protocols. Example of protocols that can be used are in vitro protocols such as specific binding to branched DNA using e.g. an electrophoretic mobility shift assay (EMSA) or enzyme-linked immunoassay, or e.g. by specific binding to Chk1 polypeptide by co-immunoprecipitation.

Alternatively, in vivo protocols can be carried out e.g. by quantitative PCR analysis of mRNA levels, immunoblotting of Claspin protein, analysis of co-immunoprecipitation of Chk1 with claspin, analysis of the rate and extent of Chk1 phosphorylation, and/or analysis of replisome stability in the presence of replicational stress either by FACS or indirect immunofluorescence microscopy

In a preferred embodiment, claspin activity is measured by extent of the recruitment of Claspin to immobilised, branched DNA. Such a measurement provides a reading from which any changes to the activity of claspin on addition of the test agent can then be compared.

The assay system can comprise a claspin polypeptide or nucleic acid which is then contacted with a test agent. Such a test agent may be any agent capable of modulating the activity and/or expression of claspin (polypeptide or nucleic acid), such as by interfering with the DNA-binding function of claspin, and is preferably a small molecule such as a chemical compound or complex thereof, an antibody, or a nucleic acid inhibitor, such as double stranded RNA, antisense oligomer, PMO or any other type of interfering RNA.

In a method of the invention, the test agent is contacted with the claspin by any suitable procedure know to the person of skill in the art. Preferable procedures are set out as follows:

(i) incorporating the test agent in a solution to a solution containing recombinant Claspin in the presence or absence of DNA, or Chk1;
(ii) incorporating the test agent in a solution to a solution containing cells expressing claspin;
(iii) introducing the test agent into cells expressing claspin by disrupting the cell membrane;
(iv) introducing the test agent into cells by first incorporating the test substance into a molecular device capable of fusion with the cell membrane (e.g. cationic liposomes).

It will be understood that any procedure that achieves the desired effect of enabling contact of the test agent with the claspin may be utilised.

The contacting of the test agent with the claspin or the cells expressing the claspin may be carried out for any suitable amount of time, preferably an amount of time that is sufficient for the test agent to have some effect on the expression and/or activity of the claspin. For example, if cells expressing claspin are used in the assay, then a greater amount of time may be required to ensure that the test agent has contacted the claspin (e.g. since the agent may have to pass through the membrane of the cell/nucleus) than if the claspin used in the assay is purified in the assay system. Typical amounts of time that the test agent may be left in the assay system is about 0 to 10 hours. For example, about 20 minutes to about 10 hours, about 20 minutes to about 2 hours, about 20 minutes to about 1 hour, about 1 hour to about 8 hours, about 2 hours to about 4 hours.

Further, the contacting of the test agent with the claspin may be carried out in conditions (e.g. temperature, pH, etc.) that are similar to the environment where the present invention will find most use, such as in conditions of an animal body, preferably a human body.

After the test agent has been left in contact with the claspin for a suitable amount of time, and under suitable conditions, the expression and/or activity of the claspin is measured again, using the same protocols as were used to measure the reference value of the claspin. In this way, a comparative result can be obtained which will show whether the function (e.g. activity and/or expression) of the claspin has been modulated. In this regard, the activity and/or expression may be up-regulated, down-regulated, or may remain unchanged. If the function remains unchanged, then it is likely that the test agent screened is not a modulator of claspin.

In the present invention, a preferable test agent is one that down-regulates claspin.

Once a modulator of claspin has been identified, then such a modulator can be used in the other embodiments of the present invention as further discussed herein.

In a preferred embodiment the method involves:
(a) contacting a first population of cells in which BRCA and claspin function is essentially normal with the test agent;
(b) contacting a second population of cells in which BRCA is abrogated but in which claspin is functional with the test agent;
(c) exposing the first and second cell populations to replicative stress; and
(d) determining the effects of said test agent on replication proliferation or survival of cells of the first and second populations.

Preferably the BRCA is BRCA1.

Where a test agent reduces proliferation or survival of the second population (i.e. where BRCA1 has been abrogated), but has a reduced or less significant effect on the first population (i.e. where BRCA1 is still functional), this indicates a test agent that is a specific inhibitor of claspin or members of its downstream pathway, but does not affect BRCA1 or its downstream pathway. Such a method therefore allows the identification of lead compounds which can potentially target cells in which BRCA1 has been abrogated, e.g. cancerous cells, while leaving cells in which BRCA1 remains functional, i.e. healthy cells. This provides the possibility for targeted therapy of cancers in which BRCA1 is deficient by utilising the synthetic lethality achieved in cells in which both claspin and BRCA1 is deficient. Synthetic lethality arises when a combination of deficiencies in two or more genes or their protein products leads to cell death, whereas a deficiency in either one does not, and by itself is said to be viable.

By BRCA and claspin function being ‘essentially normal’ or ‘functional’, it is meant that the function is such that BRCA and claspin are expressed at levels which provide a viable and functional cell.

By BRCA being ‘abrogated’ in a cell, it is meant that the function of BRCA is diminished to such an extent that the cell is effectively without BRCA function. E.g. the amount of BRCA mRNA may be diminished to such an extent as to prevent the functioning of BRCA. This may not be immediately apparent in light of the redundancy of a number of genes, proteins and pathways within a cell, but will become apparent when a factor or a combination of factors is applied to the cell such that it is no longer viable or functional without the ‘abrogated’ gene or gene product.

The person of skill in the art will be readily able to ascertain whether the function (e.g. expression and/or activity) of BRCA or claspin is ‘normal’ or ‘abrogated’ in a cell. Typical methods of ascertaining such conditions are by e.g. mRNA or protein quantification, of which the skilled person will readily understand and be able to perform.

As examples of mRNA quantification, levels of mRNA can be quantitatively measured by Northern blotting which gives size and sequence information about the mRNA molecules. Typically, a sample of RNA is separated on an agarose gel and hybridized to a radio-labeled RNA probe that is complementary to the target sequence. The radio-labeled RNA is then detected by an autoradiograph.

An alternative and low-throughput approach for measuring mRNA abundance is reverse transcription quantitative polymerase chain reaction (RT-PCR followed with qPCR). RT-PCR first generates a DNA template from the mRNA by reverse transcription (cDNA). This cDNA template is then used for qPCR where the change in fluorescence of a probe changes as the DNA amplification process progresses. With a carefully constructed standard curve qPCR can produce an absolute measurement such as number of copies of mRNA, typically in units of copies per nanolitre of homogenized tissue or copies per cell.

Alternatively, DNA microarray technology can be used to measure transcript levels for a gene. Alternatively “tag based” technologies like Serial analysis of gene expression (SAGE), which can provide a relative measure of the cellular concentration of different messenger RNAs, can be used.

As examples of protein quantification, a common method of protein quantification is to perform a Western blot against the protein of interest—this gives information on the size of the protein in addition to its identity. Typically, a sample (often cellular lysate) is separated on a polyacrylamide gel, transferred to a membrane and then probed with an antibody to the protein of interest. The antibody can either be conjugated to a fluorophore or to horseradish peroxidase for imaging and/or quantification.

An alternative method of protein quantification is e.g. the enzyme-linked immunosorbent assay (ELISA). ELISA works by using antibodies immobilised on a microtiter plate to capture proteins of interest from samples added to the well. Using a detection antibody conjugated to an enzyme or fluorophore the quantity of bound protein can be accurately measured by fluorometric or colourimetric detection. The detection process is very similar to that of a Western blot, but by avoiding the gel steps more accurate quantification can be achieved.

In certain embodiments of the present invention, it is envisaged that BRCA and/or claspin ‘normality’ will be achieved with function at about or at least 50%, 60%, 70%, 80%, 85%, 90%, 95%, 96%, 97%, 98%, 99%, 100% and over 100% of that of cell in which BRCA and/or claspin is functional, such as a HeLa cell.

In certain embodiments of the present invention, it is envisaged that a BRCA function of less than 30%, 20%, 10%, 5%, 4%, 3%, 2%, 1% and 0% as compared to a normal cell in which BRCA is functional, such as a HeLa cell, will be detrimental to the cell if BRCA function is required for specific cell functions and cell viability.

Suitable populations of cells for the first and second populations will be apparent to the person skilled in the art. Suitable cell lines in which BRCA1 has been abrogated are the BRCA-null cells HCC1937 and SUM1315MO2, although other cell lines will be apparent to the person skilled in the art. Suitable cell lines in which BRCA and claspin is functional include HeLa, U2OS cells or MCF-7, although other cell lines will be apparent to the person skilled in the art. It is routine in the art to knock-out or knock-down expression of particular genes in cells and therefore it is well within the ability of the skilled artisan to provide other cells having the desired properties.

The contacting of the first and second populations of cells with the test agent may be carried out for any suitable amount of time, preferably an amount of time that is sufficient for the test agent to have some effect on the function (e.g. expression and/or activity) of the claspin. Typical amounts of time that the test agent may be left in the assay system is about 0 to 10 hours. For example, about 20 minutes to about 10 hours, about 20 minutes to about 2 hours, about 20 minutes to about 1 hour, about 1 hour to about 8 hours, about 2 hours to about 4 hours.

Further, the contacting of cell populations with the test agent may be carried out in conditions (e.g. temperature, pH, etc.) that are similar to the environment where the present invention will find most use, such as in conditions of an animal body, preferably a human body.

After the test agent has been left in contact with the cell populations for a suitable amount of time, and under suitable conditions, the cell populations are subjected to conditions promoting replicative stress. There a variety of ways in which the cells can be exposed to replicative stress, but the most suitable for use in the present invention are chemical means or ionising radiation. A preferred agent for placing the cells under replicative stress is hydroxyurea (HU). Preferably, the means for putting the cells under replicative stress is achieved through an appropriate anti-tumour agent, such as one that may already be used in anti-cancer therapies. This may provide an advantage of assessing the likely outcomes of potential combination therapies of the test agent and anti-tumour agent which may ultimately be used in a clinical setting.

Again, the cell populations are exposed to replicative stress for an amount of time that is sufficient to halt or interfere with DNA replication. For chemical exposure, such a time may range from a matter of minutes to a number of hours.

Suitably the ability of the test substance to inhibit replication or survival is determined by identifying the number or proportion of cells within the populations in which the cell cycle has been arrested. Of particular interest are cells in which the cell cycle has been arrested in the S phase. A preferred method of investigating status of cells within the cell cycle is FACS.

It is known that the overall rate of DNA synthesis is governed by the number of active origins together with the intrinsic catalytic rate of the replication machinery operating at a replication fork. One way of analyzing replicative competence is to assess the ability of cells to incorporate halogenated deoxynucleotides, e.g. BrdU (Sigma-Aldrich), during a brief pulse as a function of their precise position within S-phase. The pulse time can range from a few minutes to a number of hours, but preferably the pulse time is in the region of about 15 to 60 minutes, preferably 20 to 40 minutes, preferably about 30 minutes. A total cell population analysis of the amount of BrdU incorporated in every cell plotted against DNA content for that cell produces a characteristic arc, allowing an accurate determination of the proportion of cells in G1, S and G2/M phases of the cell cycle.

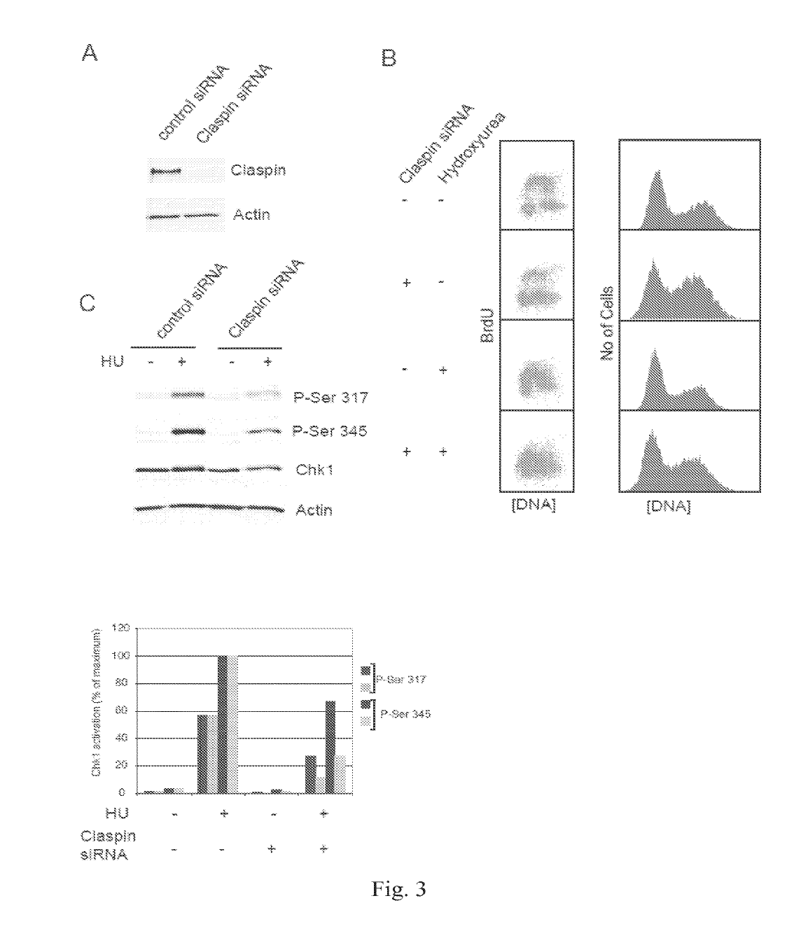

In particular, the extent of failure in replicative competence can be quantified by measuring the fraction of cells in each sample population which are in S phase as judged by their having a DNA content intermediate between G1 and G2 cells, but which are completely unable to incorporate BrdU (e.g. corresponding to Region of Interest (RoI) in FIG. 3).

In this way, the present invention may provide an assay to ascertain whether compounds are inhibitors of claspin and therefore whether those compounds may be useful in therapies (alone or e.g. in combination therapies) to treat certain disease states (e.g. cancer). The test compounds may be novel compounds (e.g. previously unknown compounds), or else they may already been known (e.g. already used in therapy). If, for example, the compounds are already known, then the present invention may provide an assay to ascertain whether those already-known molecules are inhibitors of claspin and therefore provides a potentially quick and cost-effective way of improving existing therapeutic treatments by ascertaining whether drugs that are already authorised for use on the human or animal body may be utilised in a combination therapy with e.g. anti-tumour agents to improved the efficacy of those agents in the treatment of certain disease states (e.g. cancer).

In yet another embodiment of the present invention there is provided a method, such as an in vitro method, for modulating the viability of a cell having a BRCA gene defect, said method comprising contacting the cell with an inhibitor of claspin. Preferably, the cell may be further contacted with an anti-tumour agent.

As used herein, the term “claspin polypeptide” refers to a full-length claspin protein or a functionally active fragment or derivative thereof. A claspin polypeptide can be one as referenced in, for example, Genbank Accession No. AAG24515.1. A “functionally active” claspin fragment or derivative exhibits one or more functional activities associated with a full-length, wild-type claspin protein, such as antigenic or immunogenic activity, enzymatic activity, ability to bind natural cellular substrates, etc. The functional activity of claspin proteins, derivatives and fragments can be assayed by various methods known to one skilled in the art (Current Protocols in Protein Science (1998) Coligan et al., eds., John Wiley & Sons, Inc., Somerset, N.J.) and as further discussed below. For purposes herein, functionally active fragments also include those fragments that comprise one or more structural domains of a claspin, such as a binding domain. Protein domains can be identified using the PFAM program (Bateman A., et al., Nucleic Acids Res, 1999, 27: 260-2). In some embodiments, preferred fragments are functionally active, domain-containing fragments comprising at least 25 contiguous amino acids, preferably at least 50, more preferably 75, and most preferably at least 100 contiguous amino acids of a claspin. In further preferred embodiments, the fragment comprises the entire functionally active domain.

The term “claspin nucleic acid” refers to a DNA or RNA molecule that encodes a claspin polypeptide. A claspin nucleic acid can be one as referenced in, for example, Genbank Accession No. AF297866. Preferably, the claspin polypeptide or nucleic acid or fragment thereof is from a human, but can also be an ortholog, or derivative thereof with at least 70% sequence identity, preferably at least 80%, more preferably 85%, still more preferably 90%, and most preferably at least 95% sequence identity with human claspin. Methods of identifying orthologs are known in the art. Normally, orthologs in different species retain the same function, due to presence of one or more protein motifs and/or 3-dimensional structures. Orthologs are generally identified by sequence homology analysis, such as BLAST analysis, usually using protein bait sequences. Sequences are assigned as a potential ortholog if the best hit sequence from the forward BLAST result retrieves the original query sequence in the reverse BLAST (Huynen M A and Bork P, Proc Natl Acad Sci (1998) 95: 5849-5856; Huynen M A et al., Genome Research (2000) 10: 1204-1210). Programs for multiple sequence alignment, such as CLUSTAL (Thompson J D et al, 1994, Nucleic Acids Res 22: 4673-4680) may be used to highlight conserved regions and/or residues of orthologous proteins and to generate phylogenetic trees. In a phylogenetic tree representing multiple homologous sequences from diverse species (e.g. retrieved through BLAST analysis), orthologous sequences from two species generally appear closest on the tree with respect to all other sequences from these two species. Structural threading or other analysis of protein folding (e.g. using software by ProCeryon, Biosciences, Salzburg, Austria) may also identify potential orthologs. In evolution, when a gene duplication event follows speciation, a single gene in one species, such as Drosophila, may correspond to multiple genes (paralogs) in another, such as human. As used herein, the term “orthologs” encompasses paralogs.

As used herein, “percent (%) sequence identity” with respect to a subject sequence, or a specified portion of a subject sequence, is defined as the percentage of nucleotides or amino acids in the candidate derivative sequence identical with the nucleotides or amino acids in the subject sequence (or specified portion thereof), after aligning the sequences and introducing gaps, if necessary to achieve the maximum percent sequence identity, as generated by the program WU-BLAST-2. 0al9 (Altschul et al., J. Mol. Biol. (1997) 215: 403-410) with all the search parameters set to default values. The HSP S and HSP S2 parameters are dynamic values and are established by the program itself depending upon the composition of the particular sequence and composition of the particular database against which the sequence of interest is being searched. A % identity value is determined by the number of matching identical nucleotides or amino acids divided by the sequence length for which the percent identity is being reported. “Percent (%) amino acid sequence similarity” is determined by doing the same calculation as for determining % amino acid sequence identity, but including conservative amino acid substitutions in addition to identical amino acids in the computation.

A conservative amino acid substitution is one in which an amino acid is substituted for another amino acid having similar properties such that the folding or activity of the protein is not significantly affected. Aromatic amino acids that can be substituted for each other are phenylalanine, tryptophan, and tyrosine; interchangeable hydrophobic amino acids are leucine, isoleucine, methionine, and valine; interchangeable polar amino acids are glutamine and asparagine; interchangeable basic amino acids are arginine, lysine and histidine; interchangeable acidic amino acids are aspartic acid and glutamic acid; and interchangeable small amino acids are alanine, serine, threonine, cysteine and glycine.

Alternatively, an alignment for nucleic acid sequences is provided by the local homology algorithm of Smith and Waterman (Smith and Waterman, 1981, Advances in Applied Mathematics 2: 482-489; database: European Bioinformatics Institute; Smith and Waterman, 1981, J. of Molec. Biol., 147: 195-197; Nicholas et al., 1998, “A Tutorial on Searching Sequence Databases and Sequence Scoring Methods” (www.psc.edu) and references cited therein; W. R. Pearson, 1991, Genomics 11: 635-650). This algorithm can be applied to amino acid sequences by using the scoring matrix developed by Dayhoff (Dayhoff: Atlas of Protein Sequences and Structure, M. O. Dayhoff ed., 5 suppl. 3: 353-358, National Biomedical Research Foundation, Washington, D.C., USA), and normalized by Gribskov (Gribskov 1986 Nucl. Acids Res. 14 (6): 6745-6763). The Smith-Waterman algorithm may be employed where default parameters are used for scoring (for example, gap open penalty of 12, gap extension penalty of two). From the data generated, the “Match” value reflects “sequence identity”.

Derivative nucleic acid molecules of the subject nucleic acid molecules include sequences that hybridize to the nucleic acid sequence of a claspin. The stringency of hybridization can be controlled by temperature, ionic strength, pH, and the presence of denaturing agents such as formamide during hybridization and washing. Conditions routinely used are set out in readily available procedure texts (e.g. Current Protocol in Molecular Biology, Vol. 1, Chap. 2.10, John Wiley & Sons, Publishers (1994); Sambrook et al., Molecular Cloning, Cold Spring Harbor (1989)). In some embodiments, a nucleic acid molecule of the invention is capable of hybridizing to a nucleic acid molecule containing the nucleotide sequence of a claspin under high stringency hybridization conditions that are: prehybridization of filters containing nucleic acid for 8 hours to overnight at 65 C in a solution comprising 6× single strength citrate (SSC) (1×SSC is 0.15 M NaCl, 0.015 M Na citrate; pH 7.0), 5×Denhardt's solution, 0.05% sodium pyrophosphate and 100 g/ml herring sperm DNA; hybridization for 18-20 hours at 65 C in a solution containing 6×SSC, 1×Denhardt's solution, 100 μg/ml yeast tRNA and 0.05% sodium pyrophosphate; and washing of filters at 65 C for 1 hr in a solution containing 0.1×SSC and 0.1% SDS (sodium dodecyl sulfate).

In other embodiments, moderately stringent hybridization conditions are used that are: pretreatment of filters containing nucleic acid for 6 h at 40 C in a solution containing 35% formamide, 5×SSC, 50 mM Tris-HCl (pH7.5), 5 mM EDTA, 0.1% PVP, 0.1% Ficoll, 1% BSA, and 500 μg/ml denatured salmon sperm DNA; hybridization for 18-20 h at 40 C in a solution containing 35% formamide, 5×SSC, 50 mM Tris-HCl (pH7.5), 5 mM EDTA, 0.02% PVP, 0.02% Ficoll, 0.2% BSA, 100, μg/ml salmon sperm DNA, and 10% (wt/vol) dextran sulfate; followed by washing twice for 1 hour at 55 C in a solution containing 2×SSC and 0.1% SDS.

Alternatively, low stringency conditions can be used that are: incubation for 8 hours to overnight at 37 C in a solution comprising 20% formamide, 5×SSC, 50 mM sodium phosphate (pH 7.6), 5×Denhardt's solution, 10% dextran sulfate, and 20 μg/ml denatured sheared salmon sperm DNA; hybridization in the same buffer for 18 to 20 hours; and washing of filters in 1×SSC at about 37 C for 1 hour.

As used herein, a “claspin-modulating agent” is any agent that modulates claspin function, for example, an agent that interacts with claspin to inhibit or enhance claspin activity or otherwise affect normal claspin function. Claspin function can be affected at any level, including transcription, protein expression, protein localization, and cellular or extra-cellular activity. In a preferred embodiment, the claspin-modulating agent specifically modulates the function of the claspin. The phrases “specific modulating agent”, “specifically modulates”, etc. are used herein to refer to modulating agents that directly bind to the claspin polypeptide or nucleic acid, and preferably inhibit, enhance, or otherwise alter, the function of the claspin. These phrases also encompass modulating agents that alter the interaction of the claspin with a binding partner, substrate, or cofactor (e.g. by binding to a binding partner of a claspin, or to a protein/binding partner complex, and altering claspin function). Moreover, as used herein a claspin-modulating agent can be an agent that modulates a downstream component involved in the Claspin-dependent control of the DNA replication process.

Preferably, the claspin modulating agent is an inhibitor of claspin.

Preferred claspin-modulating agents include small molecule compounds; claspin-interacting proteins, including antibodies and other biotherapeutics; and nucleic acid modulators such as antisense and RNA inhibitors. The modulating agents may be formulated in pharmaceutical compositions, for example, as compositions that may comprise other active ingredients, as in combination therapy, and/or suitable carriers or excipients. Techniques for formulation and administration of the compounds may be found in “Remington's Pharmaceutical Sciences”, Mack Publishing Co., Easton, Pa., 19th edition.

Small molecules are often preferred to modulate function of proteins with enzymatic function, and/or containing protein interaction domains. Chemical agents, referred to in the art as “small molecule” compounds are typically organic, non-peptide molecules, having a molecular weight up to 10,000, preferably up to 5,000, more preferably up to 1,000, and most preferably up to 500 daltons. This class of modulators includes chemically synthesized molecules, for instance, compounds from combinatorial chemical libraries. Synthetic compounds may be rationally designed or identified based on known or inferred properties of the claspin protein or may be identified by screening compound libraries. Alternative appropriate modulators of this class are natural products, particularly secondary metabolites from organisms such as plants or fungi, which can also be identified by screening compound libraries for claspin-modulating activity. Methods for generating and obtaining compounds are well known in the art (Schreiber S L, Science (2000) 151: 1964-1969; Radmann J and Gunther J, Science (2000) 151: 1947-1948).

Small molecule modulators identified from screening assays, as described below, can be used as lead compounds from which candidate clinical compounds may be designed, optimized, and synthesized. Such clinical compounds may have utility in treating pathologies associated with defects in BRCA. The activity of candidate small molecule modulating agents may be improved several-fold through iterative secondary functional validation, as further described below, structure determination, and candidate modulator modification and testing. Additionally, candidate clinical compounds are generated with specific regard to clinical and pharmacological properties. For example, the reagents may be derivatized and re-screened using in vitro and in vivo assays to optimize activity and minimize toxicity for pharmaceutical development.

Specific claspin-interacting proteins are useful in a variety of diagnostic and therapeutic applications related to BRCA-related disorders, as well as in validation assays for other claspin-modulating agents. In a preferred embodiment, claspin-interacting proteins affect normal claspin function, including transcription, protein expression, protein localization, and cellular or extra-cellular activity. In another embodiment, claspin-interacting proteins are useful in detecting and providing information about the function of claspin proteins, as is relevant to BRCA related disorders, such as cancer (e.g. for diagnostic means).

A claspin-interacting protein may be endogenous, i.e. one that naturally interacts genetically or biochemically with a claspin, such as a protein that modulates claspin expression, localization, and/or activity. Yeast two-hybrid and variant screens offer preferred methods for identifying endogenous claspin-interacting proteins (Finley, R. L. et al. (1996) in DNA Cloning-Expression Systems: A Practical Approach, eds. Glover D. & Hames B. D (Oxford University Press, Oxford, England), pp. 169-203; Fashema S F et al., Gene (2000) 250:1-14; Drees BL Curr Opin Chem Biol (1999) 3: 64-70; Vidal M and Legrain P Nucleic Acids Res (1999) 27: 919-29; and U.S. Pat. No. 5,928,868). Mass spectrometry is an alternative preferred method for the elucidation of protein complexes (reviewed in, e.g. Pandley A and Mann M, Nature (2000) 405: 837-846; Yates JR 3rua, Trends Genet (2000) 16: 5-8).

A claspin-interacting protein may be an exogenous protein, such as a claspin-specific antibody (see, e.g. Harlow and Lane (1988) Antibodies, A Laboratory Manual, Cold Spring Harbor Laboratory; Harlow and Lane (1999) Using antibodies: a laboratory manual. Cold Spring Harbor, N.Y.: Cold Spring Harbor Laboratory Press). Claspin-specific antibodies are further discussed below.

In preferred embodiments, a claspin-interacting protein specifically binds a claspin protein. In alternative preferred embodiments, a claspin-modulating agent binds a claspin substrate, binding partner, or cofactor.

In another embodiment, the protein modulator is a claspin-specific antibody agonist or antagonist. The antibodies have therapeutic and diagnostic utilities, and can be used in screening assays to identify claspin modulators.

Antibodies that specifically bind claspin polypeptides can be generated using known methods. Preferably the antibody is specific to a mammalian ortholog of claspin polypeptide, and more preferably, to human claspin. Antibodies may be polyclonal, monoclonal (mAbs), humanized or chimeric antibodies, single chain antibodies, Fab fragments, F(ab′)₂ fragments, fragments produced by a FAb expression library, anti-idiotypic (anti-Id) antibodies, and epitope-binding fragments of any of the above. Epitopes of claspin which are particularly antigenic can be selected, for example, by routine screening of claspin polypeptides for antigenicity or by applying a theoretical method for selecting antigenic regions of a protein (Hopp and Wood (1981), Proc. Natl. Acad. Sci. U.S.A. 78: 3824-28; Hopp and Wood, (1983) Mol. Immunol. 20: 483-89; Sutcliffe et al., (1983) Science 219: 660-66) to the amino acid sequence of a claspin. Monoclonal antibodies with affinities of 10⁸ M⁻¹ preferably 10⁹ M⁻¹ to 10¹⁰ M⁻¹, or stronger can be made by standard procedures as described (Harlow and Lane, supra; Goding (1986) Monoclonal Antibodies: Principles and Practice (2d ed) Academic Press, New York; and U.S. Pat. Nos. 4,381,292; 4,451,570; and 4,618,577).

Antibodies may be generated against crude cell extracts of claspin or substantially purified fragments thereof. If claspin fragments are used, they preferably comprise at least 10, and more preferably, at least 20 contiguous amino acids of a claspin protein. Claspin-specific antigens and/or immunogens can be coupled to carrier proteins that stimulate the immune response. For example, the subject polypeptides are covalently coupled to the keyhole limpet hemocyanin (KLH) carrier, and the conjugate is emulsified in Freund's complete adjuvant, which enhances the immune response. An appropriate immune system such as a laboratory rabbit or mouse is immunized according to conventional protocols.

The presence of claspin-specific antibodies is assayed by an appropriate assay such as a solid phase enzyme-linked immunosorbant assay (ELISA) using immobilized corresponding claspin polypeptides. Other assays, such as radioimmunoassays or fluorescent assays might also be used.

Chimeric antibodies specific to claspin polypeptides can be made that contain different portions from different animal species. For instance, a human immunoglobulin constant region may be linked to a variable region of a murine mAb, such that the antibody derives its biological activity from the human antibody, and its binding specificity from the murine fragment. Chimeric antibodies are produced by splicing together genes that encode the appropriate regions from each species (Morrison et al., Proc. Natl. Acad. Sci. (1984) 81: 6851-6855; Neuberger et al., Nature (1984) 312: 604-608; Takeda et al., Nature (1985) 31: 452-454).

Humanized antibodies, which are a form of chimeric antibodies, can be generated by grafting complementary-determining regions (CDRs) (Carlos, T. M., J. M. Harlan. 1994. Blood 84: 2068-2101) of mouse antibodies into a background of human framework regions and constant regions by recombinant DNA technology (Riechmann L M, et al., 1988 Nature 323: 323-327). Humanized antibodies contain murine sequences and human sequences, and thus further reduce or eliminate immunogenicity, while retaining the antibody specificities (Co M S, and Queen C. 1991 Nature 351: 501-501; Morrison SL. 1992 Ann. Rev. Immun. 10: 239-265). Humanized antibodies and methods of their production are well-known in the art (U.S. Pat. Nos. 5,530,101, 5,585,089, 5,693,762, and 6,180,370). Claspin-specific single chain antibodies which are recombinant, single chain polypeptides formed by linking the heavy and light chain fragments of the Fv regions via an amino acid bridge, can be produced by methods known in the art (U.S. Pat. No. 4,946,778; Bird, Science (1988) 242: 423-426; Huston et al., Proc. Natl. Acad. Sci. USA (1988) 85: 5879-5883; and Ward et al., Nature (1989) 334: 544-546).

Other suitable techniques for antibody production involve in vitro exposure of lymphocytes to the antigenic polypeptides or alternatively to selection of libraries of antibodies in phage or similar vectors (Huse et al., Science (1989) 246: 1275-1281).

Existing claspin antibodies can be used and are known to persons of skill in the art. Examples of such antibodies are e.g. catalogue nos. A300-266A, A300-265A, A300-267A and BP300-266, all from Bethyl Laboratories, Inc. (Texas, USA).

The polypeptides and antibodies of the present invention may be used with or without modification. Frequently, antibodies will be labelled by joining, either covalently or non-covalently, a substance that provides for a detectable signal, or that is toxic to cells that express the targeted protein (Menard S, et al., Int J. Biol Markers (1989) 4: 131-134). A wide variety of labels and conjugation techniques are known and are reported extensively in both the scientific and patent literature. Suitable labels include radionuclides, enzymes, substrates, cofactors, inhibitors, fluorescent moieties, fluorescent emitting lanthanide metals, chemiluminescent moieties, bioluminescent moieties, magnetic particles, and the like (U.S. Pat. Nos. 3,817,837; 3,850,752; 3,939,350; 3,996,345; 4,277,437; 4,275,149; and 4,366,241). Also, recombinant immunoglobulins may be produced (U.S. Pat. No. 4,816,567). Antibodies to cytoplasmic polypeptides may be delivered and reach their targets by conjugation with membrane-penetrating toxin proteins (U.S. Pat. No. 6,086,900).

When used therapeutically in a patient, the antibodies of the subject invention are typically administered parenterally, when possible at the target site, or intravenously. The therapeutically effective dose and dosage regimen is determined by clinical studies. Typically, the amount of antibody administered is in the range of about 0.1 mg/kg to about 10 mg/kg of patient weight. For parenteral administration, the antibodies are formulated in a unit dosage injectable form (e.g. solution, suspension, emulsion) in association with a pharmaceutically acceptable vehicle. Such vehicles are inherently nontoxic and non-therapeutic. Examples are water, saline, Ringer's solution, dextrose solution, and 5% human serum albumin. Nonaqueous vehicles such as fixed oils, ethyl oleate, or liposome carriers may also be used. The vehicle may contain minor amounts of additives, such as buffers and preservatives, which enhance isotonicity and chemical stability or otherwise enhance therapeutic potential. The antibodies' concentrations in such vehicles are typically in the range of about 1 mg/ml to about 10 mg/ml.

Immunotherapeutic methods are further described in the literature (U.S. Pat. No. 5,859,206; WO0073469).

Other preferred claspin-modulating agents comprise nucleic acid molecules, such as antisense oligomers or double stranded RNA (dsRNA), which generally inhibit claspin activity. Preferred nucleic acid modulators interfere with the function of the claspin nucleic acid such as DNA replication, transcription, translocation of the claspin RNA to the site of protein translation, translation of protein from the claspin RNA, splicing of the claspin RNA to yield one or more mRNA species, or catalytic activity which may be engaged in or facilitated by the claspin RNA.

In one embodiment, the antisense oligomer is an oligonucleotide that is sufficiently complementary to a claspin mRNA to bind to and prevent translation, preferably by binding to the 5′ untranslated region. Claspin-specific antisense oligonucleotides, preferably range from at least 6 to about 200 nucleotides. In some embodiments the oligonucleotide is preferably at least 10, 15, or 20 nucleotides in length. In other embodiments, the oligonucleotide is preferably less than 50, 40, or 30 nucleotides in length. The oligonucleotide can be DNA or RNA or a chimeric mixture or derivatives or modified versions thereof, single-stranded or double-stranded. The oligonucleotide can be modified at the base moiety, sugar moiety, or phosphate backbone. The oligonucleotide may include other appending groups such as peptides, agents that facilitate transport across the cell membrane, hybridization-triggered cleavage agents, and intercalating agents.

In one embodiment of the invention a siNA molecule comprises an antisense strand comprising a nucleotide sequence that is complementary to an mRNA sequence or a portion thereof encoding a claspin protein.

In one embodiment, the antisense region of claspin siNA constructs comprises a sequence complementary to sequence having any of SEQ ID NOs. 1, 3, 5, and 7. In one embodiment, the antisense region of claspin constructs comprises sequence having any of SEQ ID NOs. 2, 4, 6, 8, 9, 10, 11, 12 and 13.

In one embodiment of the invention, there is provided an RNA comprising, or consisting of, or consisting essentially of, a sequence of SEQ ID NO: 2, 4, 6, 8, 9, 10, 11, 12 or 13. Preferably, the sequence is one of SEQ ID NOs: 9, 10, 11, 12 or 13.

In some embodiments, there may be provided a sequence of SEQ ID NOs: 2, 4, 6, 8, 9, 10, 11, 12, or 13 with various base substitutions, additions or deletions to said sequence. Preferably any substitutions, additions or deletions will be such that the function of the RNA as an antisense molecule to the claspin mRNA will not be significantly compromised. Preferably, the number of bases to be added, deleted or substituted will be approximately only 1, 2 or 3 bases. The ability of an antisense molecule to remain a viable antisense molecule can be tested via e.g. hybridisation tests under various stringencies, as described in more detail below.

In another embodiment, the antisense oligomer is a phosphothioate morpholino oligomer (PMO). PMOs are assembled from four different morpholino subunits, each of which contain one of four genetic bases (A, C, G, or T) linked to a six-membered morpholine ring. Polymers of these subunits are joined by non-ionic phosphodiamidate intersubunit linkages. Details of how to make and use PMOs and other antisense oligomers are well known in the art (e.g. see WO99/18193; Probst J C, Antisense Oligodeoxynucleotide and Ribozyme Design, Methods. (2000) 22(3): 271-281; Summerton J, and Weller D. 1997 Antisense Nucleic Acid Drug Dev.: 7: 187-95; U.S. Pat. No. 5,235,033; and U.S. Pat. No. 5,378,841).

Alternative preferred claspin nucleic acid modulators are double-stranded RNA species mediating RNA interference (RNAi). RNAi is the process of sequence-specific, post-transcriptional gene silencing in animals and plants, initiated by double-stranded RNA (dsRNA) that is homologous in sequence to the silenced gene. Methods relating to the use of RNAi to silence genes in C. elegans, Drosophila, plants, and humans are known in the art (Fire A, et al., 1998 Nature 391: 806-811; Fire, A. Trends Genet. 15, 358-363 (1999); Sharp, P. A. RNA interference 2001. Genes Dev. 15, 485-490 (2001); Hammond, S. M., et al., Nature Rev. Genet. 2, 110-1119 (2001); Tuschl, T. Chem. Biochem. 2, 239-245 (2001); Hamilton, A. et al., Science 286, 950-952 (1999); Hammond, S. M., et al., Nature 404, 293-296 (2000); Zamore, P. D., et al., Cell 101, 25-33 (2000); Bernstein, E., et al., Nature 409, 363-366 (2001); Elbashir, S. M., et al., Genes Dev. 15, 188-200 (2001); WO0129058; WO9932619; Elbashir S M, et al., 2001 Nature 411: 494-498; Novina CD and Sharp P. 2004 Nature 430: 161-164; Soutschek J et al 2004 Nature 432: 173-178).

In one embodiment of the invention a siNA molecule comprises an antisense strand comprising a nucleotide sequence that is complementary to an mRNA sequence or a portion thereof encoding a claspin protein. Optionally, the siNA further comprises a sense strand, wherein said sense strand is complementary to said antisense strand.

In one embodiment, the antisense region of claspin siNA constructs comprises a sequence complementary to sequence having any of SEQ ID NOs. 1, 3, 5, and 7. In one embodiment, the antisense region of claspin constructs comprises sequence having any of SEQ ID NOs. 2, 4, 6, 8, 9, 10, 11, 12 and 13. In another embodiment, where siNA duplexes are provided, the sense region of claspin constructs comprises a sequence having any of SEQ ID NOs. 1, 3, 5, 7, and complementary sequences to SEQ ID NOs: 9-13. In one embodiment, a siNA molecule of the invention comprises any of SEQ ID NOs. 1-13.

In one embodiment, a siNA molecule of the invention comprises any of SEQ ID NOs. 1-13, preferably as duplexes comprising SEQ ID NOs: 1 and 2, 3 and 4, 5 and 6, 7 and 8, 9 and a complementary sequence, 10 and a complementary sequence, 11 and a complementary sequence, 12 and a complementary sequence, and 13 and a complementary sequence.

In one embodiment of the invention, there is provided an RNA comprising, or consisting of, or consisting essentially of, a sequence of SEQ ID NO: 9, 10, 11, 12 or 13. In some embodiments, the sequences comprising SEQ ID NOs: 9, 10, 11, 12 or 13 will further comprise a substantially complementary sequence that results in the formation of an RNA duplex. Such duplexes can be used for RNA interference of the expression of claspin. The skilled person will be aware of appropriate complementary sequences to SEQ ID NOs: 9, 10, 11, 12 and 13.

Nucleic acid modulators are commonly used as research reagents, diagnostics, and therapeutics. For example, antisense oligonucleotides, which are able to inhibit gene expression with exquisite specificity, are often used to elucidate the function of particular genes (see, for example, U.S. Pat. No. 6,165,790). Nucleic acid modulators are also used, for example, to distinguish between functions of various members of a biological pathway. For example, antisense oligomers have been employed as therapeutic moieties in the treatment of disease states in animals and man and have been demonstrated in numerous clinical trials to be safe and effective (Milligan J F, et al, Current Concepts in Antisense Drug Design, J Med. Chem. (1993) 36: 1923-1937; Tonkinson J L et al., Antisense Oligodeoxynucleotides as Clinical Therapeutic Agents, Cancer Invest. (1996) 14: 54-65). Accordingly, in one aspect of the invention, a claspin-specific nucleic acid modulator is used in an assay to further elucidate the role of claspin with BRCA1. In another aspect of the invention, a claspin-specific antisense oligomer is used as a therapeutic agent for treatment of BRCA-related disease states.

Preferred claspin nucleotide modulators are siRNA inhibitors having a sequence that is able to interfere with the function (e.g. activity and/or expression) of claspin. For example, the sequences shown in SEQ ID NOs: 1-13 are used to inhibit claspin. Preferably, duplexes comprising SEQ ID NOs 1 and 2; 3 and 4; 5 and 6; 7 and 8; 9 and a complementary sequence, 10 and a complementary sequence, 11 and a complementary sequence, 12 and a complementary sequence, and 13 and a complementary sequence are used to inhibit claspin. Preferably a mixture of a number of different duplexes (e.g. 2, 3 or 4 duplexes) are used. Preferably, duplexes comprising SEQ ID NOs: 9, 10, 11, 12 and 13, respectively, can also be used, as well as single strands comprising SEQ ID NOs: 9, 10, 11, 12 and 13.

Embodiments of the present invention will now be described by way of example only, with reference to the accompanying drawings, in which:

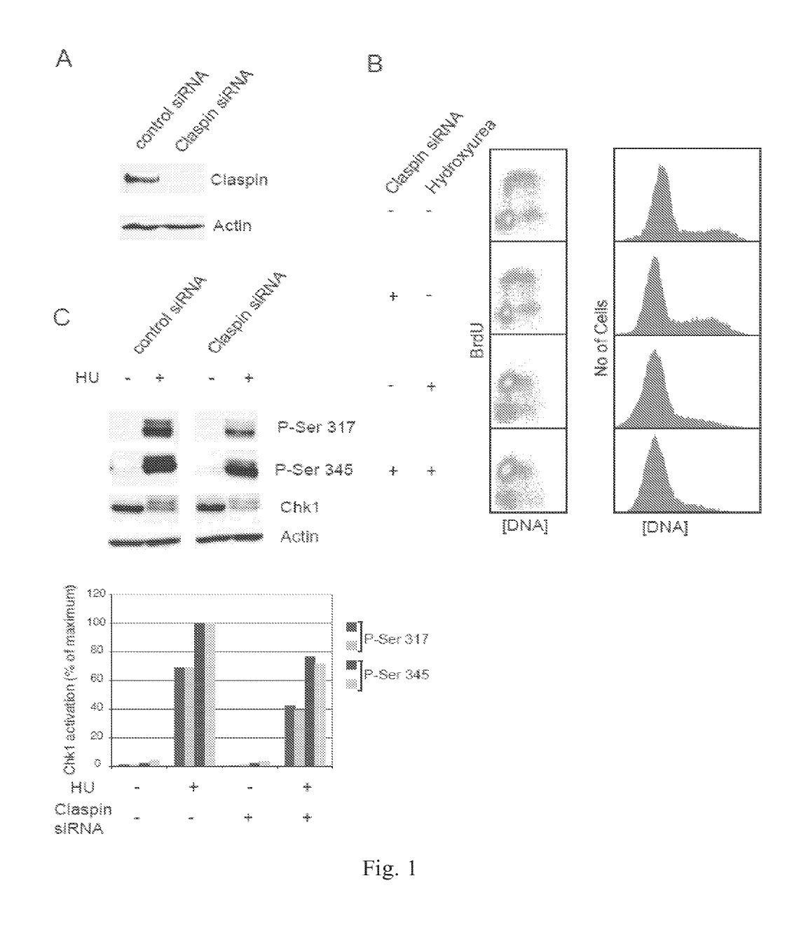

FIG. 1 shows the effect of Claspin on the cellular response to replication stress. (A) HeLa cells were transfected with control or Claspin siRNA. Cells were lysed after 48 h and subjected to immunoblotting with the indicated antibodies. (B) Cells were transfected with Claspin or control siRNA over 48 h before being treated with fresh medium or medium containing 2 mM HU (HU) for a further 24 h. Cells were subsequently incubated for 30 min in medium containing BrdU, harvested and processed for analysis by Fluorescent Activated Cell Sorting. (C) Cells transfected as in (B) above were lysed and immunoblotted with the indicated antibodies (upper panels). The blotting data was quantified using Image J software and the extent of Chk1 activation (as measured by the intensity of P-Ser345 and P-Ser 317 staining) is expressed as a proportion of either total Chk1 protein (dark columns) or total actin (light columns).

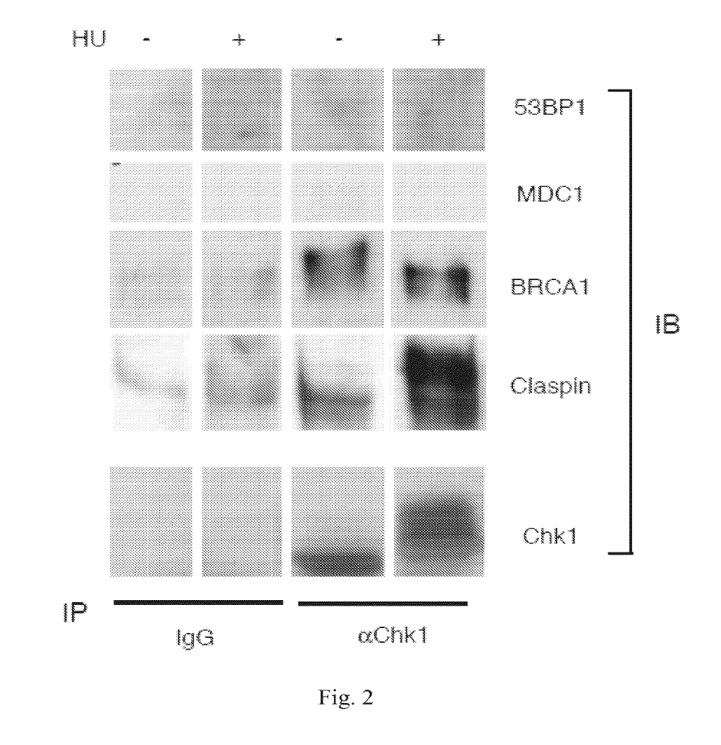

FIG. 2 shows that Chk1 forms a stable complex in vivo with BRCA1 as well as Claspin. HeLa cells were treated with 2 mM HU for 24 h prior to lysis. Cell extracts were subjected to immunoprecipitation with anti-Chk1 antibodies and the immunoprecipitates were subjected to electrophoresis prior to Immunoblotting with the indicated antibodies.

FIG. 3 shows that Claspin depletion in BRCA1-deficient HCC1937 cells results in a failure to stabilize replisomes exposed to exogenously applied replication stress. (A) HCC1937 cells were transfected with control or Claspin siRNA. Cells were lysed after 48 h and subjected to immunoblotting with the indicated antibodies. (B) Cells were transfected with Claspin or control siRNA over 48 h before being treated with fresh medium or medium containing 2 mM HU (HU) for a further 24 h. Cells were subsequently incubated for 30 min in medium containing BrdU, harvested and processed for analysis by Fluorescent Activated Cell Sorting. (C) Cells transfected as in (B) above were lysed and immunoblotted with the indicated antibodies (upper panels). The blotting data was quantified using Image J software and the extent of Chk1 activation (as measured by the intensity of P-Ser345 and P-Ser 317 staining) is expressed as a proportion of either total Chk1 protein (dark columns) or total actin (light columns).

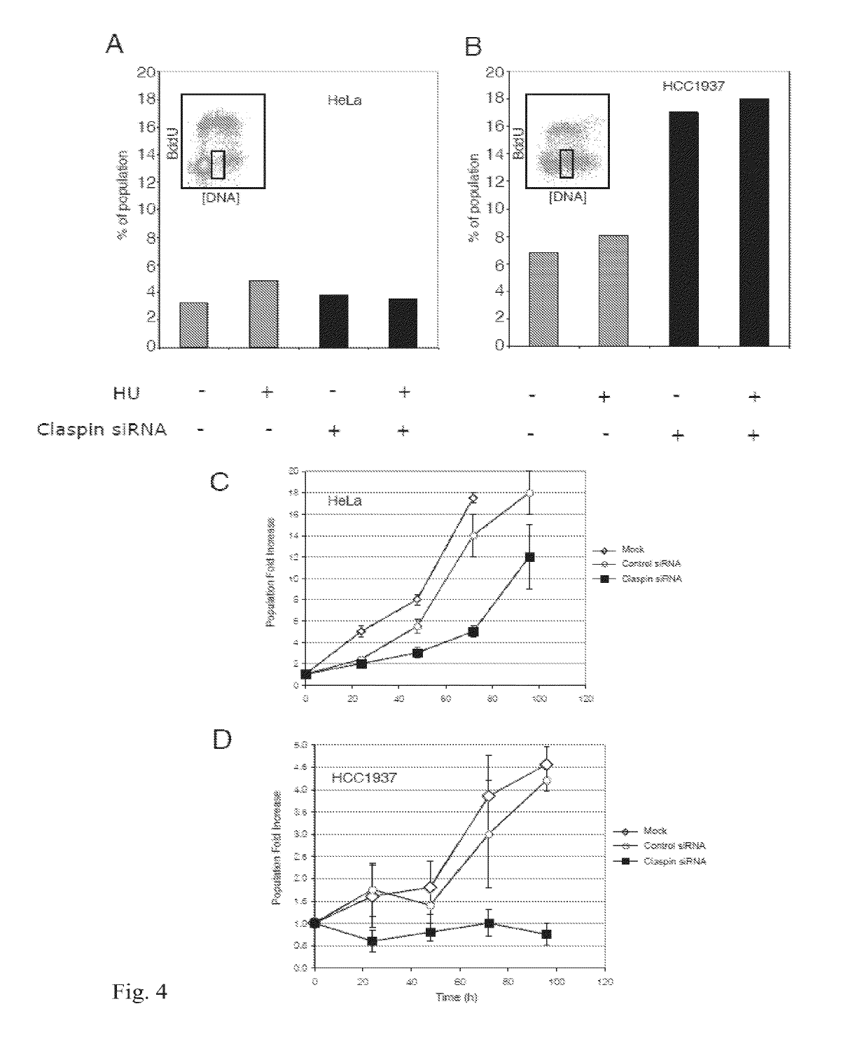

FIG. 4 shows that elevated loss of S-phase competence induced by claspin depletion in HCC1937 cells leads to significant effects on growth. HeLa cells (A) or HCC1937 cells (B) were treated with control or Claspin siRNA prior to exposure to HU for 24 h as described in the legend to FIG. 1. Cells were then incubated in medium containing BrdU for 30 min prior to processing for bivariate FACS. In each case, the proportion of S-phase cells which fail to incorporate significant levels of BrdU (as indicated by the rectangle in each insert) is plotted. (C, D) HeLa (C) or HCC1937 (D) cells were treated with control or Claspin siRNA for 48 h prior to reseeding in fresh medium. Cell numbers were determined at the indicated times and expressed as a proportion of the initial number.

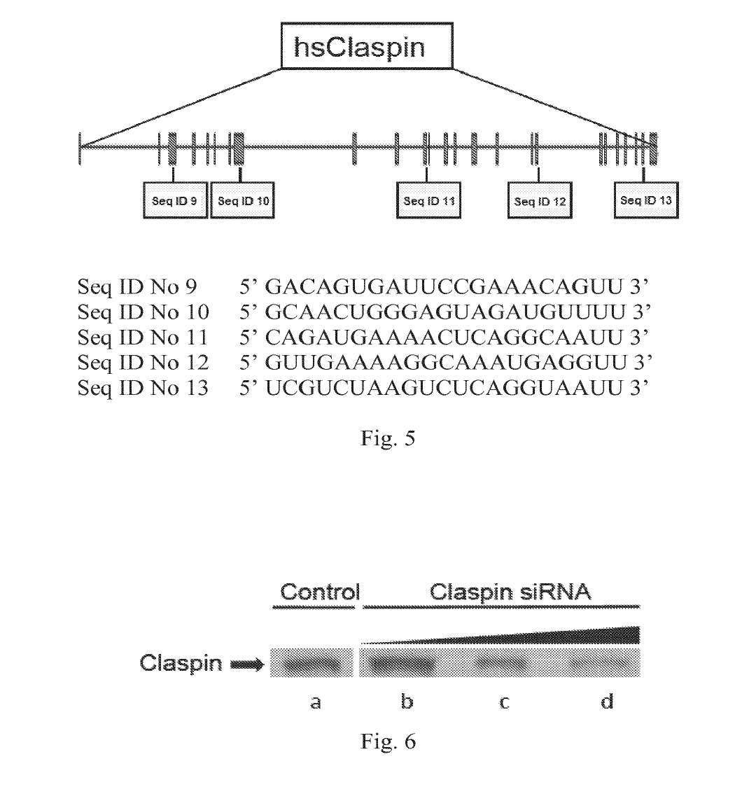

FIG. 5 shows the gene structure of human Claspin showing location of sequences selected for siRNA production and the sequences of individual siRNAs used in this study.

FIG. 6 shows a Western blot showing expression of Claspin in UWB1.289+BRCA1 (WCE) cells, a) treated with non-targeting siRNA, b) treated with 30 nM Claspin siRNA (Seq ID No 13), c) treated with 100 nM Claspin siRNA (Seq ID No 13), d) treated with 300 nM Claspin siRNA (Seq ID no 13).

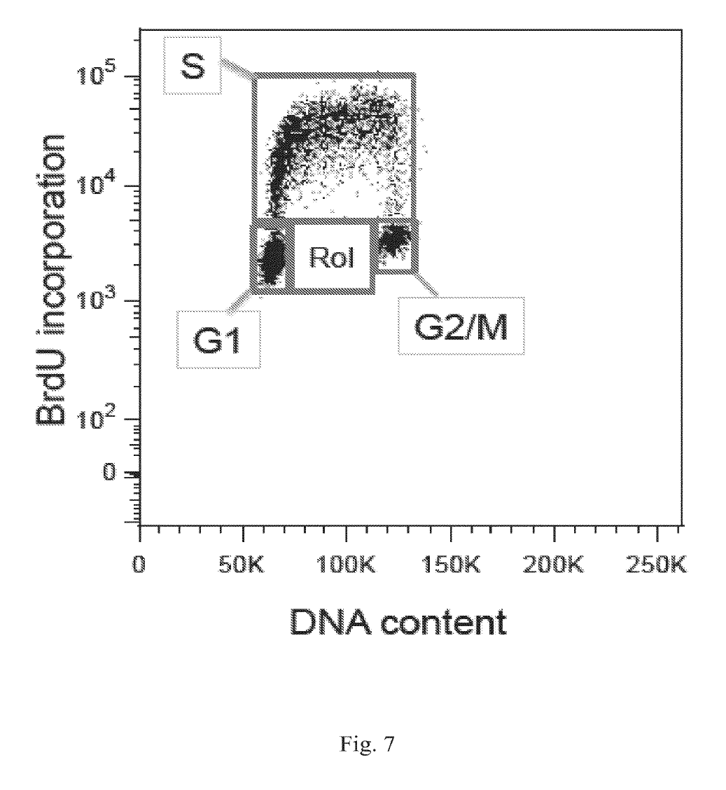

FIG. 7 shows the results of a typical bivariate FACS analysis of cervical carcinoma cell line plotting BrdU incorporation against DNA content. Cells in G1,S and G2/M phases are indicated by red rectangles. Replicative failure results in cells appearing in the region of interest (RoI).

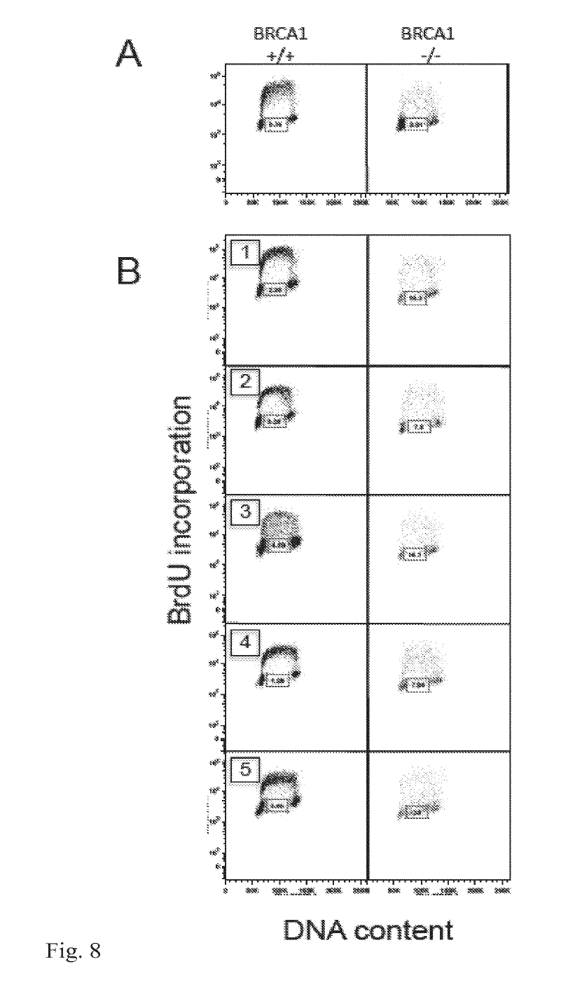

FIG. 8 shows the results of bivariate FACS analysis of (BRCA1+/+) cervical carcinoma cell line (A and B, left hand panels) and (BRCA1−/−−) breast cancer line (A and B, right hand panels) plotting BrdU incorporation against DNA content. Cells in G1,S and G2/M phases are indicated by red rectangles. Replicative failure results in cells appearing in the region of interest (RoI).

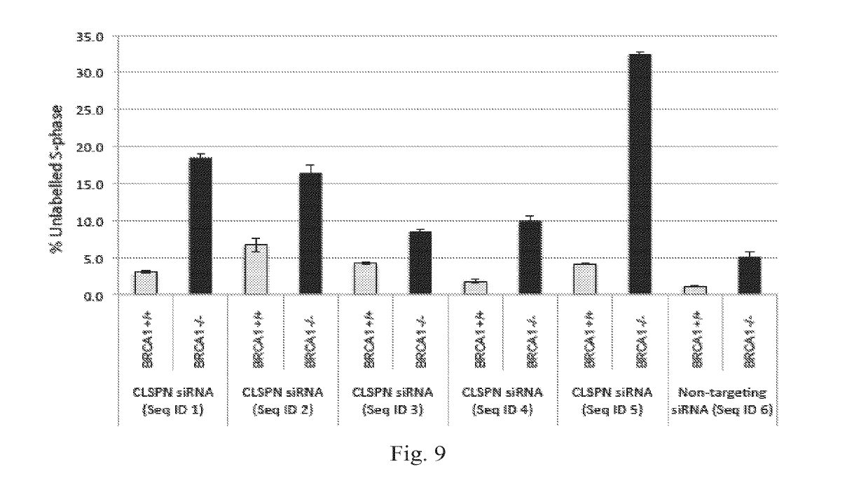

FIG. 9 shows a graph derived from the data shown in FIG. 8 showing the proportion of unlabelled S-phase cells in BRCA1+/+ (grey bars) and BRCA−/− cells (black bars) treated with Claspin siRNA (Seq ID No 9-13), or non-targeting.

The invention provides methods to identify claspin-modulating agents that are candidate therapeutic agents that can be used in the treatment of disorders associated with defective BRCA (e.g. BRCA1 or BRCA2) genes. Preferred claspin-modulating agents can specifically bind to claspin polypeptides and inhibit claspin function. Other preferred claspin-modulating agents are nucleic acid modulators such as interfering RNA (RNAi) and antisense oligomers that are able to repress claspin gene expression or product activity by, for example, binding to and inhibiting the respective nucleic acid (i.e. mRNA or DNA). Moreover, as used herein a claspin-modulating agent can be an agent that modulates a downstream component involved in the Claspin-dependent control of the DNA replication process.

Claspin modulating agents may be evaluated by any convenient in vitro or in vivo assay for molecular interaction with a claspin polypeptide or nucleic acid. In one embodiment, candidate claspin modulating agents are tested with an assay system comprising a claspin polypeptide or nucleic acid. Agents that produce a change in the activity of the assay system relative to controls are identified as candidate claspin modulating agents. The assay system may be cell-based or cell-free. Claspin-modulating agents include claspin related proteins (e.g. dominant negative mutants, and biotherapeutics); claspin-specific antibodies; claspin-specific antisense oligomers and other nucleic acid modulators; and chemical agents that specifically bind to or interact with claspin or compete with claspin binding partner (e.g. by binding to a claspin binding partner).

The invention further provides methods for modulating the claspin function in a cell (preferably a mammalian cell) by contacting the (mammalian) cell with an agent that specifically binds a claspin polypeptide or nucleic acid. The agent may be a small molecule modulator, a nucleic acid modulator, or an antibody and may be administered to a (mammalian) animal predetermined to have a pathology associated with the BRCA gene.

The invention provides methods to identify agents that interact with and/or modulate the function of claspin. Modulating agents identified by the methods are also part of the invention. Such agents are useful in a variety of diagnostic and therapeutic applications associated with BRCA gene defects, as well as in further analysis of the claspin protein and its contribution to the treatment of BRCA-related diseases. Accordingly, the invention also provides methods for modulating the viability of a cell comprising the step of specifically modulating claspin activity by administering a claspin-interacting or -modulating agent.

The invention provides assay systems and screening methods for identifying specific modulators of claspin activity. As used herein, an “assay system” encompasses all the components required for performing and analyzing results of an assay that detects and/or measures a particular event. In general, primary assays are used to identify or confirm a modulator's specific biochemical or molecular effect with respect to the claspin nucleic acid or protein.

In a preferred embodiment, the screening method comprises contacting a suitable assay system comprising a claspin polypeptide or nucleic acid with a candidate agent under conditions whereby, but for the presence of the agent, the system provides a reference activity, which is based on the particular molecular event the screening method detects. A statistically significant difference between the agent-biased activity and the reference activity indicates that the candidate agent modulates claspin activity. The claspin polypeptide or nucleic acid used in the assay may comprise any of the nucleic acids or polypeptides described above.

Specific claspin-modulating agents are useful in a variety of diagnostic and therapeutic applications where disease or disease prognosis is related to defects in BRCA, such as angiogenic, apoptotic, or cell proliferation disorders.

The invention also provides methods for treating disorders or disease associated with impaired BRCA function by administering a therapeutically effective amount of a claspin-modulating agent in combination with an anti-tumour agent. The invention further provides methods for modulating claspin function in a cell, preferably a cell pre-determined to have defective or impaired BRCA function, by administering a claspin-modulating agent. Additionally, the invention provides a method for treating disorders or disease associated with impaired BRCA function by administering a therapeutically effective amount of a claspin-modulating agent.

The invention includes pharmaceutical compositions for the treatment of abnormal cell growth in a mammal, including a human.

In one embodiment, the abnormal cell growth is cancer, particularly a cancer that involves malignant cells which have a defect in BRCA. As used herein, the term “cancer” unless otherwise indicated, refers to diseases that are characterized by uncontrolled, abnormal cell growth and/or proliferation. In one aspect of the invention, the cancer comprises a solid tumor including, but not limited to, metastatic solid tumors. In one aspect the solid tumor is an endothelial cell carcinoma, including, but not limited to, renal cell carcinoma, colon carcinoma, transitional cell carcinoma, lung carcinoma, breast carcinoma and prostatic carcinoma. Examples of renal cell carcinoma include, but are not limited to, clear cell carcinoma, papillary carcinoma, chromophobe carcinoma, collecting duct carcinoma and unclassified carcinoma. Examples of lung carcinoma include, but are not limited to, adenocarcinoma, alveolar cell carcinoma, squamous cell carcinoma, large cell and small cell carcinoma. Examples of breast carcinoma include, but are not limited to, adenocarcinoma, ductal carcinoma in situ, lobular carcinoma in situ, invasive ductal carcinoma, medullary carcinoma and mucinous carcinoma. In another aspect of the invention, the solid tumor is an endothelial cell sarcoma, including but not limited to, soft tissue sarcoma.

In certain embodiments, the cancerous or malignant condition may include a condition selected from the group comprising, but not limited to: malignant melanoma, chronic myelogenous leukaemia, hairy cell leukaemia, multiple myeloma, renal cell carcinoma, hepatocellular carcinoma, colorectal cancer, gastric cancer, head and neck cancer, osteosarcoma, breast cancer, ovarian cancer, cervical cancer, prostate cancer, pancreatic cancer, uterine cancer, and Non-Hodgkin's lymphoma.

As used herein, the terms “chemotherapeutic agent”, “anti-tumour agent”, “cytotoxic agent” are used interchangeably and unless otherwise indicated, refer to any agent used in the treatment of cancer which inhibits, disrupts, prevents or interferes with abnormal cell growth and/or proliferation. Examples of chemotherapeutic agents include, but are not limited to, agents which induce apoptosis, alkylating agents, purine antagonists, pyrimidine antagonists, plant alkaloids, intercalating antibiotics, aromatase inhibitors, anti-metabolites, mitotic inhibitors, growth factor inhibitors, cell cycle inhibitors, enzymes, topoisomerase inhibitors, biological response modifiers, steroid hormones and anti-androgens. In some embodiments, a monoclonal antibody can be combined with a single species of chemotherapeutic agent while in other embodiments, it can be combined with multiple species of chemotherapeutic agents.

Examples of alkylating agents include, but are not limited to, carmustine, lomustine, cyclophosphamide, ifosfamide, mechlorethamine and streptozotocin. Examples of antibiotics include, but are not limited to, adriamycin, bleomycin, dactinomycin, daunorubicin, doxorubicin, idarubicin and plicamycin. Examples of anti-metabolites include, but are not limited to, cytarabine, fludarabine, 5-fluorouracil, 6-mercaptopurine, methotrexate and 6-thioguanine. Examples of mitotic inhibitors include, but are not limited to, navelbine, paclitaxel, vinblastine and vincristine. Examples of steroid hormones and anti-androgens include, but are not limited to, aminoglutethimides, estrogens, flutamide, goserelin, leuprolide, prednisone and tamoxifen.

In some embodiments of the invention the cytotoxic agent is selected from the group consisting of cytotoxins, chemotherapeutic agents and radiation. Examples of cytotoxins include but are not limited to, gelonin, ricin, saponin, pseudomonas exotoxin, pokeweed antiviral protein, diphtheria toxin and complement proteins. Examples of chemotherapeutic agents include but are not limited to, alkylating agents, purine antagonists, pyrimidine antagonists, plant alkaloids, intercalating antibiotics, aromatase inhibitors, anti-metabolites, mitotic inhibitors, growth factor inhibitors, cell cycle inhibitors, enzymes, topoisomerase inhibitors, biological response modifiers, anti-hormones and anti-androgens. Additional examples of chemotherapeutic agents include but are not limited to, BCNU, cisplatin, gemcitabine, hydroxyurea, paclitaxel, temozomide, topotecan, fluorouracil, vincristine, vinblastine, procarbazine, dacarbazine, altretamine, cisplatin, methotrexate, mercaptopurine, thioguanine, fludarabine phosphate, cladribine, pentostatin, fluorouracil, cytarabine, azacitidine, vinblastine, vincristine, etoposide, teniposide, irinotecan, docetaxel, doxorubicin, daunorubicin, dactinomycin, idarubicin, plicamycin, adriamycin, mitomycin, bleomycin, tamoxifen, flutamide, leuprolide, goserelin, aminoglutethimide, anastrozole, amsacrine, asparaginase, mitoxantrone, mitotane and amifostine.

In yet another embodiment where the cytotoxic agent is radiation, the radiation is a radioisotope. Examples of radioisotopes include, but are not limited to, ³H, ¹⁴C, ¹⁸F, ¹⁹F, ³¹P, ³²P, ³⁵S, ¹³¹I, ¹²⁵I, ¹²³I, ⁶⁴Cu, ¹⁸⁷Re, ¹¹¹In, ⁹⁰Y, ^{99m}Tc, ¹⁷⁷Lu. In one embodiment, the radioisotope is linked to an antibody by α-(5-isothiocyanato-2-methoxyphenyl)-1,4,7,10-tetraazacyclododecane-1,4,7,10-tetraacetic acid (methoxy-DOTA). Another example of radiation is external beam radiation.

Examples of pharmaceutical formulations of the above chemotherapeutic agents include, but are not limited to, BCNU (i.e., carmustine, 1,3-bis(2-chloroethyl)-1-nitrosurea, BiCNU®), cisplatin (cis-platinum, cis-diamminedichloroplatinum, Platinol®), doxorubicin (hydroxyl daunorubicin, Adriamycin®), gemcytabine (difluorodeoxycytidine, Gemzar®), hyrdoxyurea (hyroxycarbamide, Hydrea®), paclitaxel (Taxol®), temozolomide (TMZ, Temodar®), topotecan (Hycamtin®), fluorouracil (5-fluorouracil, 5-FU, Adrucil®), vincristine (VCR, Oncovin®) and vinblastine (Velbe® or Velban®).

Pharmaceutical compositions of the present invention can be administered via parenteral, subcutaneous, intravenous, intramuscular, intraperitoneal, transdermal or buccal routes. For example, an agent may be administered locally to a tumor via microinfusion. Alternatively, or concurrently, administration may be by the oral route. For example, a chemotherapeutic agent could be administered locally to the site of a tumor, followed by oral administration of at least one agent which modulates claspin. The process can be carried out in reverse (e.g. the claspin-modulating agent is administered locally to the site of the tumour whilst a therapeutic agent is administered e.g. intravenously). The administration of the chemotherapeutic agent followed or preceded by the claspin modulator may have the effect of reducing the amount of chemotherapeutic agent necessary in subsequent treatments for successful outcomes, thus reducing the severe side effects associated with chemotherapeutic agents. The dosage administered will be dependent upon the age, health, and weight of the recipient, kind of concurrent treatment, if any, frequency of treatment, and the nature of the effect desired.

In addition, the compositions of the present invention may contain suitable pharmaceutically acceptable carriers comprising excipients and auxiliaries that facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil or synthetic fatty acid esters, for example, ethyl oleate or triglycerides. Aqueous injection suspensions may contain substances which increase the viscosity of the suspension include, for example, sodium carboxymethyl cellulose, sorbitol and dextran. Optionally, the suspension may also contain stabilizers. Liposomes can also be used to encapsulate the agent for delivery into the cell. The pharmaceutical formulation for systemic administration according to the invention may be formulated for enteral, parenteral or topical administration. Indeed, all three types of formulations may be used simultaneously to achieve systemic administration of the active ingredient.

As mentioned above, topical administration may be used. Any common topical formulation such as a solution, suspension, gel, ointment or salve and the like may be employed. Preparation of such topical formulations are described in the art of pharmaceutical formulations as exemplified, for example, by Gennaro et al. (1995) Remington's Pharmaceutical Sciences, Mack Publishing. For topical application, the compositions could also be administered as a powder or spray, particularly in aerosol form. In some embodiments, the compositions of this invention may be administered by inhalation. For inhalation therapy the active ingredients may be in a solution useful for administration by metered dose inhalers or in a form suitable for a dry powder inhaler. In another embodiment, the compositions are suitable for administration by bronchial lavage.

Suitable formulations for oral administration include hard or soft gelatin capsules, pills, tablets, including coated tablets, elixirs, suspensions, syrups or inhalations and controlled release forms thereof. In another embodiment, the pharmaceutical composition comprises the claspin modulator in combination with at least one cytotoxic agent wherein the modulator or agent are in sustained release form. In such formulations, the claspin modulator will be distributed throughout the body, prior to, or after release of the cytotoxic agents. In one embodiment, upon the delayed release of the claspin modulator from such formulations, and subsequent distribution to the site of the cancer cells, the effects of the modulator may be enhanced by the earlier binding or effect of the cytotoxic agent on the cancer cells. Such delayed release formulations may have the same effect as sequential administration of one or more cytotoxic agents followed by the claspin modulator or vice versa.

As used herein and unless otherwise indicated, the term “pharmaceutically acceptable” means approved by a regulatory agency for use in animals, and more particularly in humans. The term “vehicle” refers to a diluent, adjuvant, excipient, or carrier with which a compound of the invention is administered. Such pharmaceutical vehicles can be, for example, liquids, such as water and oils, including those of petroleum, animal, vegetable or synthetic origin, such as peanut oil, soybean oil, mineral oil, sesame oil and the like. The pharmaceutical vehicles can be saline, methyl cellulose, gum acacia, gelatin, starch paste, talc, keratin, colloidal silica, urea, and the like. In addition, auxiliary, stabilizing, thickening, lubricating and coloring agents may be used. When administered to a patient, the compositions of the invention and pharmaceutically acceptable vehicles are preferably sterile. Water is a preferred vehicle when the composition of the invention is administered intravenously. Saline solutions and aqueous dextrose and glycerol solutions can also be employed as liquid vehicles, particularly for injectable solutions.

Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

As used herein and unless otherwise indicated, the phrase “pharmaceutically acceptable salt” includes, but is not limited to, salts of acidic or basic groups that may be present in compositions. Polypeptides included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, (i.e., salts containing pharmacologically acceptable anions), including, but not limited to, sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e. 1,1′-methylene-bis-(2-hydroxy-3-naphthoate) salts. Polypeptides included in compositions used in the methods of the invention that are acidic in nature are capable of forming base salts with various pharmacologically acceptable cations. Examples of such salts include alkali metal or alkaline earth metal salts and, particularly, calcium, magnesium, sodium lithium, zinc, potassium, and iron salts.

As used herein and unless otherwise indicated, the term “therapeutically effective” refers to an amount of a claspin modulator and/or cytotoxic agent or a pharmaceutically acceptable salt, solvate or hydrate thereof able to cause an amelioration of a disease or disorder, or at least one discernible symptom thereof. “Therapeutically effective” also refers to an amount that results in an amelioration of at least one measurable physical parameter, not necessarily discernible by the patient. In yet another embodiment, the term “therapeutically effective” refers to an amount that inhibits the progression of a disease or disorder, either physically (e.g., stabilization of a discernible symptom), physiologically (e.g., stabilization of a physical parameter), or both. In yet another embodiment, the term “therapeutically effective” refers to an amount that results in a delayed onset of a disease or disorder.

As used herein and unless otherwise indicated, the term “prophylactically effective” refers to an amount of a claspin modulator, cytotoxic agent or a pharmaceutically acceptable salt, solvate or hydrate thereof causing a reduction of the risk of acquiring a given disease or disorder. In one embodiment, the compositions are administered as a preventative measure to an animal, preferably a human, having a genetic predisposition to a disorder described herein. In another embodiment of the invention, the compositions are administered as a preventative measure to a patient having a non-genetic predisposition to a disorder disclosed herein. The compositions of the invention may also be used for the prevention of one disease or disorder and concurrently treating another.

This invention includes methods for the treatment of cancer in a mammal, including a human. Such methods include the treatment or inhibition of abnormal growth and/or proliferation of cancer cells including malignant cells of neoplastic diseases. Inhibition of abnormal cell growth can occur by a variety of mechanisms including, but not limited to, apoptosis, cell death, inhibition of cell division, transcription, translation, transduction, etc. As discussed above, claspin modulators can be provided in combination, or in sequential combination with cytotoxic agents that are useful in the treatment of cancer. As used herein, two agents are said to be administered in combination when the two agents are administered simultaneously or are administered independently in a fashion such that the agents will act in an additive or synergistic fashion. For example, claspin modulators can be used in combination with one or more chemotherapeutic agents selected from the following types of chemotherapeutic agents including, but not limited to, apoptotic agents, mitotic inhibitors, alkylating agents, antimetabolites, intercalating antibiotics, growth factor inhibitors, cell cycle inhibitors, enzymes, topoisomerase inhibitors, biological response modifiers, anti-hormones, and anti-androgens as described herein.

In practicing the methods of this invention, a claspin modulator may be used alone or in combination with other therapeutic or diagnostic agents. In certain preferred embodiments, the claspin modulator may be co-administered along with other chemotherapeutic agents typically prescribed for various types of cancer according to generally accepted oncology medical practice. The compositions of this invention can be utilized in vivo, ordinarily in mammals, such as humans, sheep, horses, cattle, pigs, dogs, cats, rats and mice or in vitro. The invention is particularly useful in the treatment of human subjects.

Co-administration can be separate, sequential or combined. Thus, the claspin modulator may be administered separately either before or after the anti-tumour agent. If the claspin modulator is administered before the anti-tumour agent, this may be advantageous in that it may ‘prime’ the cells to be more susceptible to the anti-tumour agent. Thus there may be a reduced dose of anti-tumour agent required to achieve a therapeutic effect, which is beneficial to the well being of the patients as the side-effects of the anti-tumour agent may be reduced. Conversely, if the anti-tumour agent is administered before the claspin modulator, then the cells may be more amenable to the claspin modulator, resulting in a faster kill-time.

Alternatively, the co-administration may be combined either in one pharmaceutical preparation or in two separate preparations taken at the same time.

In particular embodiments of the present invention, there is provided a combination of an anti-claspin siRNA molecule and an agent that targets DNA replication, such as an anticancer agent as discussed above, e.g. a chemotherapeutic agent or cytotoxic agent.

Any anti-claspin RNA molecule may be suitable, and can be easily constructed by a person of skill in the art using well known techniques, e.g. ascertaining a particular sequence within the claspin gene and constructing an appropriate antisense molecule to the mRNA which would be produced from that DNA sequence.

In particular, there is provided a combination of an RNA molecule comprising one or more of SEQ ID NOs: 1, 2, 3, 4, 5, 6, 7, 8, 9, 10, 11, 12, and 13 and an anticancer agent, preferably SEQ ID NOs: 2, 4, 6, 8, 9, 10, 11, 12 and 13 and an anticancer agent. Yet further, there is provided a combination of an RNA duplex comprising SEQ ID NOs: 1 and 2, 3 and 4, 5 and 6, 7 and 8, 9 and a complementary sequence, 10 and a complementary sequence, 11 and a complementary sequence, 12 and a complementary sequence, and 13 and a complementary sequence, and an anticancer agent.

Preferably the combination of an siRNA molecule and anti-replication agent is used for the treatment of a proliferative disease such as cancer resulting from a defect in the BRCA1 or BRCA2 genes As discussed above, such combination therapy can be one where the siRNA is administered prior to, at the same time as, or after the anti-replication agent.

A number of so-called adaptor proteins have been implicated in the cellular response to replication stress and have been proposed to act either upstream or directly to facilitate the activation of the Chk1 pathway. The present inventors have shown previously that Chk1 is a signalling component of an intra S-phase replisome stability checkpoint which ensures that pathways which sense arrested replication forks and ionising radiation induced double strand breaks and that one function of Chk1 is to ensure that activation of late-firing replication origins is blocked when synthesis from origins firing early in S phase is inhibited. Claspin was originally identified in Xenopus egg extracts as a Chk1 binding protein which was required for the phosphorylation and activation of Chk1 by ATR. The present inventors have been investigating the role of Claspin in maintaining replisome stability following imposition of replication stress.

In order to investigate a role for Claspin in the replisome stability checkpoint, siRNA (see SEQ ID NOs: 1-13) and siRNA duplexes (e.g. SEQ ID NOs: 1 and 2; 3 and 4; 5 and 6; 7 and 8 etc.) were used to deplete Claspin from HeLa cells.

Treatment of HeLa cells with a specific siRNA resulted in >90% depletion of Claspin after 48 hours compared to cells exposed to control siRNA (e.g. luciferase, which is not expressed in these cells) (FIG. 1A). In order to evaluate the effect of Claspin depletion on replisome stability, the ability of Claspin-depleted cells to maintain functional replisomes in the continuous presence of imposed replicational stress was investigated (FIG. 1). To do this, cells were treated with buffer control or 2 mM hydroxyurea (HU) for 24 h, then exposed to a 30 min pulse of BrdU, prior to fixation and PI staining, and subsequent bivariate FACS analysis. In the absence of any imposed replication stress, abrogation of Claspin had little or no effect on the ability of otherwise untreated cells to undergo S-phase (FIG. 1B, left-hand panels) or to traverse the cell cycle (FIG. 1B, right-hand panels). Exposure of cells to hydroxyurea for 24 hr results in a significant accumulation of cells in early S phase as expected (FIG. 1B). Interestingly Claspin-depleted cells were capable of incorporating BrdU at a rate comparable to that obtained in untreated cells suggesting that the absence of Claspin alone has no gross effect on the number of functional replication forks even after 24 h exposure to HU (FIG. 1B).

These data suggest that the replisome stability checkpoint remains intact in the almost complete absence of Claspin. Consistent with this observation, the present inventors have found that signalling through Chk1, as measured by the degree of Chk1 phosphorylation at ser317 and ser345, expressed either as a fraction of the amount of total Chk1, or the amount of activated Chk1 per cell, was only slightly affected (20-40%) by the absence of Claspin (FIG. 1C).

In order to determine whether any other adaptor proteins play a role in this checkpoint, the inventors first sought to establish which of the known adaptor proteins could be found in stable complexes with Chk1 by co-immunoprecipitation. Many studies have demonstrated the existence of such complexes in cells transfected with, and over-expressing, tagged proteins; the present inventors sought to investigate the existence of endogenous complexes in untransfected cells. Chk1 was immunoprecipitated from asynchronously growing or HU-treated cells and immunoprecipitates were probed for the presence of adaptor proteins implicated in various aspects of DNA damage responses. As expected, Claspin could be detected in Chk1 IPs (FIG. 2). Interestingly, the amount of Chk1-associated Claspin was significantly increased in HU-treated cells. The present inventors also found BRCA1 in Chk1 immunoprecipitates, although the levels of BRCA1 were unaffected by HU treatment. Other BRCT domain-containing proteins such as MDC1 or TopBP1 were not detected in Chk1 immunoprecipitates (FIG. 2).

BRCA1 has been implicated previously in the cellular response to IR, and BRCA1-deficient cells fail to activate Chk1 following IR exposure. The inventors investigated the effect of siRNA-mediated knockdown of Claspin in HCC1937 cells which lack functional BRCA1 and which have been previously shown to have a defective G2 checkpoint, failing to arrest on exposure to IR. Treatment of HCC1937 cells with Claspin siRNA consistently resulted in >90% loss of Claspin (FIG. 3A). Interestingly, the additional loss of Claspin resulted in some increase in G2 cells (FIG. 3B) as analysed by FACS, suggesting that these cells undergo some delay in G2 even in the absence of any exogenous stress. Exposure of mock siRNA-treated HCC1937 cells to hydroxyurea resulted in accumulation of cells in early S-phase as expected (FIG. 3B). Importantly, HCC1937 cells remained largely capable of incorporating BrdU even after 24 hr exposure to HU, suggesting that the absence of BRCA1 also has no gross effect on the number of functional replication forks even after 24 h exposure to HU. In sharp contrast, ablation of Claspin in HCC1937 cells resulted in dramatic alteration in the bivariate FACS profile of cells exposed to HU (FIG. 3B, left-hand panels), with significant proportion of S-phase cells failing to incorporate BrdU. The loss of BrdU incorporation was particularly observed in mid- to late-S phase cells.

The present inventors have shown previously that ablating Chk1 function results in loss of an intra S-phase checkpoint regulating replisome stability and origin firing in mammalian cells. The activation state of Chk1 in mock and Claspin siRNA-treated HCC1937 cells exposed to HU was determined (FIG. 3C), as measured by the degree of Chk1 phosphorylation at ser317 and ser345 determined by immunoblotting. It was found that in mock siRNA-treated cells, 24 hr exposure to HU resulted in significant activation of Chk1 (FIG. 3C) with the extent of Chk1 phosphorylation comparable to that observed in cells containing wild-type BRCA1 (FIG. 1). Interestingly, ablation of Claspin in HCC1937 cells resulted in a similar reduction in phosphorylated Chk1 (FIG. 3C), when expressed as a fraction of total Chk1 protein as that observed in cells lacking Claspin alone (FIG. 1). However, the absence of Claspin and BRCA1 function combined resulted in significant (>75%) reduction of the levels of activated Chk1 per cell (FIG. 3C).

Taken together, the data in FIGS. 1-4 suggest that BRCA1 and Claspin act co-ordinately in maintaining the stability of replisomes under conditions of imposed replication stress, and that they do so by activating the Chk1 protein kinase.

As all cells are believed to experience some replication stress during normal progression through S-phase, it is conceivable that loss of both BRCA1 and Claspin would result in significant levels of replisome instability during S-phase, resulting in incomplete replication of the genome. The present inventors therefore investigated the significance of the combined loss of BRCA1 and Claspin function on cell proliferation. In order to do this, HeLa or HCC1937 cells were exposed to either control or Claspin siRNA as described previously, and cells were plated under normal growth conditions and cell numbers were determined at subsequent times. HCC1937 cells are known to have a doubling time ˜30 h and so proliferate significantly more slowly than HeLa cells (FIGS. 4A &B). Exposure of HeLa cells to Claspin siRNA resulted in a slight decrease in growth characteristics with a population increase of 12-fold compared to 18-fold in control siRNA-treated cells after 96 h culture. Mock-treated HCC1937 cells showed approximately 3-fold population increase over the same period. Interestingly, HCC1937 cells lacking Claspin failed to show any significant proliferation with net cell numbers remaining the same even after 96 h. These data indicate that the presence of either functional BRCA1 or Claspin is sufficient for cell viability. However, cells lacking both genes fail to proliferate.

This data, together with previously published work, indicate that BRCA1 and Claspin form complexes with Chk1 in vivo and co-operate to facilitate the stabilization of replication machinery in the presence of replication stress. In addition the present inventors have found while loss of Claspin reduces the efficiency with which Chk1 becomes phosphorylated in the presence of HU, the lack of BRCA1 as well as Claspin results in both reduced efficiency of signalling and reduced levels of Chk1 protein, resulting in significant reduction in effective Chk1 signalling during replication stress.

Taken together, the results are consistent with a model in which both BRCA1 and Claspin co-operate to facilitate the phosphorylation and activation of a specific sub-fraction of the total cellular Chk1 protein, which in turns acts to stabilize components of the replication machinery when ongoing replication is blocked as a consequence of endogenous or exogenously applied replication stress. The mechanism by which arrested replication forks are stabilized as well as the process by which Claspin and BRCa1 facilitate the activation of a specific fraction of cellular Chk1 to bring this about remains unknown.

Chk1 protein kinase has been the subject of considerable interest as a potential target for the development of novel anti-cancer therapeutics as it plays a role in multiple cell cycle checkpoint responses. Several potent inhibitors of Chk1 (such as UCN-01, which bind to its ATP binding site) have been reported. However, their lack of complete specificity and significant overall cytotoxicity make them somewhat unlikely lead compounds for novel therapeutic applications.

The results reported here provide a novel opportunity for the development of new therapeutics for individuals who have a genetic predisposition to the development of cancer, such as breast cancer. Such individuals have inherited one functional and one non-functional form of either BRCA1 or BRCA2. During the process of transformation which will lead ultimately to the onset of the disease, cells emerge which have lost the second, functional form of the relevant BRCA gene. We have found that the breast cancer cell line HCC1937 in which we have experimentally ablated Claspin function and which lack both copies of BRCA1 is profoundly sensitive to antitumour agents, such as agents which temporarily interfere with DNA replication (this being the basis of much chemotherapeutic intervention). The data indicate that in such circumstances, these cells are inviable, fail to undergo any further proliferation and ultimately die. In contrast, cells which retain either Claspin, or a normal copy of BRCA1, are relatively insensitive to such treatment.

It follows that, e.g. in hereditary breast cancer patients, a drug “cocktail” comprising anti-tumour agents (such as inhibitors of DNA replication) in combination with a specific Claspin inhibitor would be expected to have a high therapeutic index, by selectively blocking the proliferation of those cells that lack both copies of BRCA1 (i.e. in a tumour) while having minimal effects on healthy surrounding breast and other tissues, which retain one functional copy of the BRCA1 gene.

### EXAMPLES

### Cell Culture

HeLa cells, or HEK293T cells, were maintained in DMEM (Invitrogen) supplemented with 10% foetal calf serum at 37° C. and 5% CO₂. To maintain cell culture at sub-maximal confluency cell cultures were passaged 1 in 8, using trypsin (Invitrogen) twice every 7 days. HCC1937 cells were maintained in RPMI 1640 (http://www.atcc.org/Portals/1/Pdf/30-2001.pdf) supplemented with 10% foetal calf serum. To maintain cell culture at sub-maximal confluency cell cultures were passaged 1 in 8, using trypsin (Invitrogen) twice every 7 days. Cell proliferation was assayed by cell counts. HeLa or HCC1937 cells were seeded into 60 mm tissue culture plates (2×10⁵/plate) and then left for 24 hours before being treated with either 0 or 2 mM HU (Sigma-Aldrich, St Louise, Mo., USA) for up to 96 hours with media being changed every 24 hours. Cells were trypsinized with 500 μL of trypsin, resuspended in 1 mL of PBS supplemented with 5% foetal calf serum and counted in duplicate with a haemocytometer.

### Transient Transfections

HeLa or HCC1937 cells were seeded at 5×10⁴ or 2×10⁵ cells into 6-well tissue culture plates respectively, in 1 mL of their media and left for 24 hours. Cells were then washed twice with warm PBS and transfected twice over 48 hours with SMARTpool™ siRNA against Claspin (Dharmacon, Chicago, Ill., USA) at a concentration of 100 nM for 5 hours in serum free media (OptiMEM (Invitrogen)) using Oligofectamine (Invitrogen) as per manufacturer's instructions. After each transfection, cells were then washed once with warm PBS, and 1.5 mL of media added and then left for 19 hours. After transfection cells were trypsinized and seeded into 60 mm dishes and left in fresh media for 24 hours before being treated with either 0 or 2 mM HU for 24 hours. Alternatively cells were untreated, left in a flow cabinet for an equivalent time. After treatment, media was aspirated and cells were re-fed with fresh media before being further incubated at 37° C. and 5% CO₂ for 1 hour before harvesting (see below).

### Cell Cycle Analysis

Parental HeLa or HCC1937 cells were plated into 100 mm dishes (5×10⁵/plate) and 24 hours later treated with either 0 or 2 mM HU for 24 hours. 30 mins before harvesting, 25 μM BrdU (Sigma-Aldrich) was added to the media. To harvest, the cells were trypsinized and centrifuged at 1000 rpm at room temperature for 5 mins. The supernatant was then removed and the cells were resuspended in 10 mL of IFA buffer (150 mM Tris-HCl pH 7.6, 500 mM NaCl, 0.5% NaN₃, 5% FCS). Cells were then respun at 1000 rpm, 4° C. for 5 mins before being washed twice in ice cold PBS and fixed with 70% EtOH (Sigma-Aldrich). Cells were then stained using rat anti-BrdU antibody (Abcam, Cambridge, UK) at a dilution of 1 in 50 for 2 hours at 4° C., and anti-rat IgG linked to horseradish peroxidase at a dilution of 1 in 50 for 1 hour at 4° C. Subsequently cells were DNA stained using 20 μg/mL propidium iodide (Sigma-Aldrich) and treated with 200 μg/mL RNAase A (Sigma-Aldrich) before being assayed on a DAKO Cytometer FACS machine. FACS files were collected and assayed using Summit v4.3. Cells treated with siRNA were transfected and treated as stated above before being harvested for FACS analysis using the same protocol as for parental cell lines. Additionally siRNA transfected cells were assayed for siRNA knockdown of target genes, through immunoblotting, before being assayed by FACS.

### Immunoblotting and Immunoprecipitation

HeLa or HCC1937 cells were seeded into 100 mm tissue culture dishes (5×10⁵/plate) and left for 24 hours at 37° C. and 5% CO₂. Cells were then treated with 0 or 2 mM HU for 24 hours before being harvested. Alternatively cells were untreated (left in a flow cabinet for an equivalent time). Cells were washed in twice in ice cold PBS, before being harvested in IPLB (20 mM Tris/Acetate pH 7.5, 0.27 M sucrose, 1 mM EGTA, 1 mM EDTA, 1 mM sodium orthovanadate, 10 mM sodium β-glycerophosphate, 5 mM sodium pyrophosphate, 1% (w/v) Triton X-100, 0.1% (v/v) β-Mercaptoethanol, 1 μM microcystin, 0.2 mM PMSF with a protease inhibitor cocktail (Roche, Mannheim, GER), by being scraped into Eppendorf tubes on ice. Cells were then lysed through 3 freeze/thaw cycles, and clarified by centrifugation at 14,000 rpm for 5 mins at 4° C. The supernatant was then removed and assayed for concentration by Bradford assay (Pierce) and aliquots stored at −70° C. until use. For immunoblotting 25-60 μg of whole cell extract was used per well in Laemmli SDS loading buffer. Extracts were separated on 6-12% SDS-PAGE gels before being transferred to nitrocellulose membrane (Protran) for probing. Membranes were blocked in either 5% Marvel milk or 5% BSA according to antibody manufacturer's guidelines. Membranes were then washed before being probed with primary antibodies diluted in blocking buffer. Primary antibodies used were: BRCA1=MS110 (Merck) or C20 (Santa Cruz), Claspin=BL73 (Bethyl), TopBP1=C33 (BD Biosciences), Actin=Clone AC15 (Sigma-Aldrich), Chk1=Sheep polyclonal antibody (described in Feijoo et al., 2001) or BL235 (Bethyl), PhosphoChk1 Serine 296 & 345=rabbit antibodies (Cell Signalling Technologies), PhosphoChk1 Serine 317=polyclonal rabbit antibody (Bethyl), Nucleolin=C23 (Santa Cruz), CtIP/RBBP8=C1913 or C1914 (Bethyl). Secondary antibodies were anti-rabbit IgG-HRP, anti-sheep IgG-HRP and anti-mouse IgG-HRP (Jackson Immunologicals) and bands were detected using enhanced chemiluminescence (ECL) (Amersham Pharmacia) and autoradiographic film (FUJI film).

Immunoprecipitation (IP) as carried out with 500 μg to 1 mg of whole cell extract per IP. Extracts were pre-cleared with 5 μg non-specific IgG (same species as IP antibody) (Santa Cruz) and excess of washed & equilibrated Protein-G Sepharose beads (CR-UK) for a total of 3 hours at 4° C. Extracts were then spun at 14,000 rpm for 30 seconds at 4° C., and the supernatants transferred to new chilled Eppendorf tubes. The required IP antibody was then added (up to 3 μg) and 30 μL of Protein-G Sepharose beads and incubated over night at 4° C. in a total volume of IPLB of 500-1000 μL. Beads were then spun down at 14,000 rpm for 30 seconds at 4° C. and washed once with IPLB containing 0.5M NaCl, and twice with standard IPLB before eluting the protein complexes with ×2 Laemmli SDS loading buffer (100 mM Tris-HCl pH 6.8, 4% SDS, 0.2% bromophenol blue, 20% Glycerol, 200 mM DTT) and heating at 100° C. for 5 minutes. Complexes isolated were then analysed by immunoblotting.

### Further Experimental Work

The present inventors set out to further establish a method to identify molecules which selectively interfere with Claspin-mediated replicative competence. Molecules which interfere directly with Claspin or its specific downstream effectors would be predicted to cause an increase in replicative failure in proliferating cells lacking BRCA1 compared with cells that express it. To validate this approach, the present inventors have developed an assay which utilises comparative bivariate fluorescent activated cell sorting (FACS) analysis to quantify and compare changes in replicative competence in cells. In order to demonstrate the effectiveness of such an approach, bioinformatics has been used to identify regions within the Claspin coding sequence for which the corresponding putative small interfering RNAs (siRNAs) are predicted to induce knockdown of Claspin expression and used this method to identify siRNAs with potential for therapeutic efficacy.

There is now described a methodology to identify siRNAs targeting Claspin which induce a failure in replicative competence in proliferating cells lacking BRCA1, but not cells which express it.

Materials and methods

Cell Culture: HeLa cells were maintained in DMEM (Invitrogen) supplemented with 10% foetal calf serum at 37° C. and 5% CO₂. To maintain cell culture at sub-maximal confluency, cell cultures were passaged by trypsinising (Invitrogen) at 70-80% confluency. HCC1937 cells were maintained in RPMI 1640 (Invitrogen) supplemented with 10% foetal calf serum, 2 mM Lglutamine, 1.5 g/L sodium bicarbonate, 10 mM HEPES, 1.0 mM sodium pyruvate and 4.5 g/L glucose (Sigma-Aldrich). To maintain cell culture at sub-maximal confluency cell cultures were passaged by trypsinising (Invitrogen) at 70-80% confluency. UWB1.289+BRCA1 cells, were maintained in 50% RPMI-1640 and 50% MEGM (Lonza, w/o Gentamicin/Amphoteracin B) supplemented with 200 mg/L G418 and 3% foetal calf serum at 37° C. and 5% CO₂. To maintain cell culture at sub-maximal confluency cell cultures were passaged 1 in 4, using 0.05% trypsin/EDTA (Invitrogen) twice every 7 days. Cell proliferation was assayed by cell counts. Transient Transfections: Cells were seeded in 6 well tissue culture plates in 2 mL of their media, at appropriate densities to reach 60-70% confluency in 24 hours (seeding densities optimized for each cell line, data not shown). After 24 hours the cells were washed twice with warm PBS and transfected twice over 48 hours with indicated siRNA (Dharmacon) targeting Claspin (see siRNA identification) at a concentration of 100 nM for 5 hours in serum-free media (OptiMEM (Invitrogen)) using Oligofectamine (Invitrogen) as per manufacturer's instructions. After each transfection, cells were then washed twice with warm PBS, and 2 mL of the respective media added and then left for 19 hours. After transfection cells were trypsinized and seeded into 60 mm dishes and left in fresh media for 48 hours.

Cell Cycle Analysis: Following transfection as set out above, cells were plated into 100 mm dishes (5×10⁵/plate). After 48 hours, cells were washed into fresh medium and pulsed with 25 mM BrdU (Sigma-Aldrich) for 30 mins. To harvest, the cells were then trypsinized and centrifuged at 1000 rpm at room temperature for 5 mins. The supernatant was then removed before the cells were resuspended in 10 mL of IFA buffer (150 mM Tris-HCl pH 7.6, 500 mM NaCl, 0.5% NaN₃, 5% FCS). Cells were then recentrifuged at 1000 rpm, 4° C. for 5 mins before being washed twice in ice-cold PBS and fixed with 70% EtOH (Sigma-Aldrich). Cells were then stained using rat anti-BrdU antibody (Abcam) at a dilution of 1 in 50 for 2 hours at 4° C., and anti-rat IgG linked to horseradish peroxidase at a dilution of 1 in 50 for 1 hour at 4° C.

Subsequently cells were stained for DNA using 20 mg/mL propidium iodide (Sigma-Aldrich) and treatment with 200 mg/mL RNAase A (Sigma-Aldrich) before being assayed on a LSRII (Beckton Dickinson) cytometer. FACS files were collected and assayed using FlowJo ver.8.8.6 (TreeStar Inc). Western Blot Analysis: Following transfection as set out above, plates were placed on ice and washed twice in ice cold before being lysed by IP lysis buffer (20 mM Tris Acetate pH 7.5, 0.27M Sucrose, 1 mM EDTA, 1 mM EGTA, 1 mM sodium orthovanadate, 10 mM sodium β-glycerophosphate, 5 mM sodium pyrophosphate, 1% Triton X-100, 0.1% β-mercaptoethanol, 1 μM microcystin, 0.2 mM PMSF, Protease Inhibitor Cocktail, EDTA-free). The cells were further lysed by 3 freeze-thaw cycles, and debris removed by centrifugation at 14,000 rpm at 4° C. for 5 min, and the material was quantified by Bradford assay.

### Results and Discussion

In order to identify regions within the Claspin mRNA coding sequence to which putative siRNAs are predicted to bind with high specificity and binding affinity, the AsiDesigner algorithm (http://sysbio.kribb.re.kr:8080/AsiDesigner/menuDesigner.jsf) was utilised which enables user input of threshold values for GC content and repetitive sequences, and which calculates relative binding energies to facilitate putative siRNA selection. Regions were initially ranked according to statistically weighted overall performance score. Although splice variants of Claspin have not been reported, the inventors wished to ensure that sequences selected for investigation targeted multiple exons with significant dispersion across the reported mRNA transcript. The exon structure, regions selected for siRNA design and the resultant siRNA sequences selected are shown in FIG. 5.

siRNAs corresponding to the sequences listed in FIG. 5 were introduced into a variety of cell lines including a cervical carcinoma cell line (containing wild-type BRCA1) and a breast cancer cell line (BRCA1-null) as described in Materials and Methods. The extent of Claspin protein knockdown was assessed by immunoblotting of cell lysates harvested ˜60 h after initial transfection (FIG. 6, and data not shown).

The overall rate of DNA synthesis is governed by the number of active origins together with the intrinsic catalytic rate of the replication machinery operating at a replication fork. To analyze replicative competence, the inventors investigated the ability of cells to incorporate halogenated deoxynucleotides during a brief pulse as a function of their precise position within S-phase. A total cell population analysis of the amount of BrdU incorporated in every cell plotted against DNA content for that cell produces a characteristic arc (FIG. 7), allowing an accurate determination of the proportion of cells in G1, S and G2/M phases of the cell cycle.

The extent of failure in replicative competence was quantified by measuring the fraction of cells in each sample population which are in S phase as judged by their having a DNA content intermediate between G1 and G2 cells, but which are completely unable to incorporate BrdU (corresponding to Region of Interest (RoI) in FIG. 7). In cells not exposed to any stress, this fraction varies depending on cell growth conditions but usually corresponds to ˜3-4% of cells (FIGS. 8 and 9). Analysis of replicative competence in cells transfected with siRNAs revealed that, compared to non-targeting controls, siRNAs targeting Claspin resulted in a substantial increase in the extent of replicative competence failure in BRCA1-null cells compared to cells that express BRCA1.

All siRNAs targeting Claspin showed significant selective induction of replicative failure in BRCA1-null cells. The results strongly support the notion that the selective inhibition of Claspin in cells lacking BRCA1 can induce significant failure in replicative competence, and that the comparative analysis of replicative competence by this procedure has significant potential for the identification of novel compounds that selectively target Claspin-mediated control of replicative competence for development as potential therapeutics.

Various modifications and variations to the described embodiments of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

1. An in vitro method of identifying a claspin modulating agent which does not significantly inhibit BRCA function, or an agent that modulates a downstream component involved in the Claspin-dependent control of the DNA replication process but which does not significantly inhibit BRCA function, said method comprising the steps of:
(a) contacting a first population of cells in which BRCA and claspin function is essentially normal with the test agent;
(b) contacting a second population of cells in which BRCA is abrogated but in which claspin is functional with the test agent;
(c) exposing the first and second cell populations to replicative stress; and
(d) determining the effects of said test agent on replication proliferation or survival of cells of the first and second populations.

2. The method of claim 1, wherein the BRCA is BRCA1.

3. The method of claim 1, wherein the replicative stress is achieved via chemical means.

4. The method of claim 1, wherein the replicative stress is achieved by an agent which interferes with DNA replication, preferably an anti-tumour agent.

5. The method of claim 1, wherein the first population of cells comprises cells of the lines HeLa, U2OS cells or MCF-7.

6. The method of claim 1, wherein the second population of cells comprises cells of the lines HCC1937 and SUM1315MO2.

7. The method of claim 1, wherein step (d) involves pulsing the first and second cell populations with a halogenated deoxynucleotide followed by FACS analysis.

8. The method of claim 7, wherein the FACS analysis is comparative bivahate FACS.

9. The method of claim 8, wherein said comparative bivahate FACS is used to measure the extent of failure in replicative competence to quantify the fraction of cells in each sample population which are in S phase as judged by their having a DNA content intermediate between G1 and G2 cells, but which are unable to incorporate BrdU.

10. A purified inhibitor of claspin.

11. The purified inhibitor of claspin of claim 10, wherein the inhibitor is a claspin-specific antibody or a claspin-specific nucleic acid modulator.

12. The purified inhibitor of claspin of claim 11, wherein the inhibitor comprises a claspin-specific nucleic acid modulator as shown in any one Of SEQ ID NOs: 1 to 13.

13. The inhibitor of claim 12, wherein the nucleic acid modulator comprises SEQ ID NOs: 2, 4, 6, 8, 9, 10, 11, 12 or 13, or a combination thereof.

14. The inhibitor of claim 13, wherein the nucleic acid modulator comprises SEQ ID NOs: 9, 10, 11, 12 or 13, or a combination thereof.

15. The inhibitor of claim 13, wherein the nucleic acid modulator is a duplex of SEQ ID NOs: 1 and 2, 3 and 4, 5 and 6, 7 and 8, 9 and a complementary sequence, 10 and a complementary sequence, 11 and a complementary sequence, 12 and a complementary sequence, and 13 and a complementary sequence or a combination thereof.

16. A modulator of claspin for use in the treatment of a cell proliferative disorder.

17. A modulator of claspin in combination with at least one agent which targets DNA replication for use in the treatment of a cell proliferative disorder.

18. The modulator of claim 17, wherein the combination of the modulator of claspin with the anti-replication agent during said treatment is combined, sequential or simultaneous.

19. The modulator of claim 17, wherein the agent which targets DNA replication is an anti-tumour agent.

20. The modulator of claim 16, wherein the cell proliferative disorder is associated with impaired BRCA function.

21. The modulator of claim 20, wherein said BRCA is BRCA1.

22. The modulator of claim 16, wherein the cell proliferative disorder is cancer.

23. The modulator of claim 16, wherein said modulator is a claspin-specific antibody or a claspin-specific nucleic acid modulator.

24. The modulator of claim 16, wherein said modulator modulates the activity or expression of claspin.

25. The modulator of claim 16, wherein the modulator is an inhibitor.

26. The modulator of claim 16, wherein said modulator is a nucleic acid modulator and said nucleic acid modulator is an RNA inhibitor or an antisense oligomer.

27. The modulator of claim 16, for use in the treatment of an animal predetermined to have cancer.

28. The modulator of claim 16, wherein the modulator is an inhibitor comprising any one of SEQ ID NOs: 1 to 13.

29. The modulator of claim 28, wherein the nucleic acid modulator comprises SEQ ID NOs: 2, 4, 6, 8, 9, 10, 11, 12 or 13, or a combination thereof.

30. The modulator of claim 29, wherein the nucleic acid modulator comprises SEQ ID NOs: 9, 10, 11, 12 or 13, or a combination thereof.

31. The modulator of claim 29, wherein the nucleic acid modulator comprises a duplex of SEQ ID NOs: 1 and 2, 3 and 4, 5 and 6, 7 and 8, 9 and a complementary sequence, 10 and a complementary sequence, 11 and a complementary sequence, 12 and a complementary sequence, and 13 and a complementary sequence or a combination thereof.

32. A pharmaceutical composition comprising a modulator of claspin.

33. The pharmaceutical composition of claim 32, further comprising an agent which targets DNA replication.

34. The pharmaceutical composition of claim 32, further comprising a physiologically acceptable excipient or adjuvant.

35. The modulator of claim 16 or the pharmaceutical composition comprising a modulator of claspin, wherein said treatment or agent which targets DNA replication comprises an agent selected from the group consisting of: methotrexate, 5-fluorouracil, fluorodeoxyuhdine, cytosine arabinoside, 6-mercaptopuhne, 6-thioguanine, mechloroethamine, cyclophosphamide, ifosfamide, melphalan, chlorambucil, thiotepa, mitomycin C, azihdinylbenzoquinone (AZQ), busulfan, carmustine (BCNU), lomustine (CCNU), fotemustine, carboplatin, daunorubicin, doxorubicin or adhamycin, epirubicin, dactinomycin or actinomycin D, mitoxanthrone, amsacrine, tenoposide, etoposide, ihnotecan, topotecan, vincristine, vinblastine, vindesine, vinorelbine, taxol, taxotere, and mixtures thereof.


SEQUENCE LISTING (13 sequences)

SEQ ID NO: 1 - RNA, 21 nt, Artificial Sequence
  5′-3′ Sense sequence
gcagauggguucuuaaauguu

SEQ ID NO: 2 - RNA, 21 nt, Artificial Sequence
  Antisense sequence
cauuuaagaacccaucugcuu

SEQ ID NO: 3 - RNA, 21 nt, Artificial Sequence
  5′-3′ Sense sequence
gaguagauguuuccauuaauu

SEQ ID NO: 4 - RNA, 21 nt, Artificial Sequence
  Antisense sequence
uuaauggaaacaucuacucuu

SEQ ID NO: 5 - RNA, 21 nt, Artificial Sequence
  5′-3′ Sense sequence
gcagauaguccuucagauauu

SEQ ID NO: 6 - RNA, 21 nt, Artificial Sequence
  Antisense sequence
uaucugaaggacuaucugcuu

SEQ ID NO: 7 - RNA, 21 nt, Artificial Sequence
  5′-3′ Sense sequence
gaagacaggcucacugcuauu

SEQ ID NO: 8 - RNA, 21 nt, Artificial Sequence
  Antisense sequence
uagcagugagccugucuucuu

SEQ ID NO: 9 - RNA, 21 nt, Artificial Sequence
  RNA sequence
gacagugauuccgaaacaguu

SEQ ID NO: 10 - RNA, 21 nt, Artificial Sequence
  RNA sequence
gcaacugggaguagauguuuu

SEQ ID NO: 11 - RNA, 21 nt, Artificial Sequence
  RNA sequence
cagaugaaaacucaggcaauu

SEQ ID NO: 12 - RNA, 21 nt, Artificial Sequence
  RNA sequence
guugaaaaggcaaaugagguu

SEQ ID NO: 13 - RNA, 21 nt, Artificial Sequence
  RNA sequence
ucgucuaagucucagguaauu